(19) United States
(12) Patent Application Publication
(10) Pub. No.: US 2025/0051758 A1
(43) Pub. Date: Feb. 13, 2025

(54) FolA Selection Assay to Identify Strains with Increased Soluble Target Protein Expression

(71) Applicant: ABSCI CORPORATION, Vancouver, WA (US)

(72) Inventor: Jia Liu, Vancouver, WA (US)

(21) Appl. No.: 18/721,315

(22) PCT Filed: Dec. 20, 2022
(86) PCT No.: PCT/US2022/081988

§ 371 Date: Jun. 18, 2024

Related U.S. Application Data
(60) Provisional application No. 63/292,329, filed on Dec. 21, 2021.

(51) Int. Cl. C12N 15/10; C12N 15/70; C12Q 1/18

(52) U.S. Cl. CPC C12N 15/1079; C12N 15/70; C12Q 1/18

(57) ABSTRACT

The present disclosure provides compositions and methods for identifying cells or strains that produce soluble protein. The present disclosure further provides a growth/no growth selection strategy to identify E. coli strains in a mixed population (library) that produce higher soluble protein.

### INCORPORATION BY REFERENCE OF MATERIAL SUBMITTED ELECTRONICALLY

The Sequence Listing, which is a part of the present disclosure, is submitted concurrently with the specification as a text file. The name of the text file containing the Sequence Listing is “57387_Seqlisting.txt”, which was created on Nov. 15, 2022 and is 1,893 bytes in size. The subject matter of the Sequence Listing is incorporated herein in its entirety by reference.

### BACKGROUND

Antibiotic resistance genes are commonly used as fusion partners for selection assays. Commonly used selectable fusion partners are antibiotic resistance genes. However, these methods suffer from limited linear response range where the expression of a modest amount of the antibiotic resistance gene confers nearly the full extent of practically achievable concentrations of the antibiotic. Protocols that aim to overcome this limitation employ an indirect selection scheme such as translational coupling. To date, an assay that allows a direct correlation between the expression of the antibiotic resistance gene and the expression of the soluble protein has not been provided. The present disclosure addresses this unmet need.

### SUMMARY

In one embodiment, the present disclosure provides a method of identifying a host cell capable of producing a soluble protein of interest, said method comprising the steps of: (a) preparing a population of host cells, wherein each host cell of the population comprises an expression construct capable of expressing a fusion protein comprising (i) a protein of interest and (ii) a selection protein; (b) incubating the host cells of (a) under conditions that allow expression of the fusion protein, wherein said conditions comprise a growth substance comprising at least 2 synergistic selection agents; and (c) visualizing host cells that are capable of growth; thereby identifying host cells capable of producing a soluble protein of interest.

In another embodiment, a method of identifying host cells that produce the highest amounts of a soluble protein of interest among a population of host cells is provided, said method comprising the steps of: (a) preparing a population of host cells, wherein each host cell of the population comprises an expression construct capable of expressing a fusion protein comprising (i) a protein of interest and (ii) a selection protein; (b) incubating the host cells of (a) under conditions that allow expression of the fusion protein, wherein said conditions comprise a growth substance comprising at least 2 synergistic selection agents; and (c) visualizing host cells that are capable of growth; thereby identifying host cells that produce the highest amounts of a soluble protein of interest among a population of host cells.

In some embodiments, an aforementioned method is provided, further comprising the step of binning the host cells based on the amount of soluble protein produced. In some embodiments, wherein the selection protein is a target of an antibiotic or an antibiotic resistance protein. In one embodiment, the selection protein is FolA. In another embodiment, the FolA is from E. coli. In still another embodiment, the FolA is set out in SEQ ID NO: 1.

In still other embodiments, an aforementioned method is provided comprising 2 synergistic selection agents are used, wherein the synergistic selection agents are selected from the group consisting of antibiotics, sugars, chemical agents, enzyme substrate analogs, enzyme inhibitors, agents that sequester biomolecules, chelating agents, and agents that compromise the cell wall or cell membrane. In one embodiment, the 2 synergistic selection agents are trimethoprim and sulfamethoxazole. In some embodiments, an aforementioned method is provided wherein the protein of interest is a heterologous protein. In another embodiment, the heterologous protein is selected from the group consisting of an antibody, a Fab, a scFv, a nanobody, a T cell receptor, and a chimeric antigen receptor, a growth factor, a cytokine, a hormone, an enzyme, or a functional fragment thereof.

In some embodiments, an aforementioned method is provided wherein the population of host cells comprises a library of host cells, wherein said library is comprised of cells with unique genotypes and/or that are uniquely genetically engineered. In one embodiment, the library comprises approximately one thousand to approximately one billion host cells.

In some embodiments, an aforementioned method is provided wherein the host cells are selected from the group consisting of eukaryotic cells, prokaryotic cells, bacterial cells, mammalian cells and insect cells. In another embodiment, the bacteria cells are E. coli cells. In still another embodiment, the E. coli cells comprise: (a) an alteration of gene function of at least one gene encoding a transporter protein for an inducer of at least one inducible promoter; (b) a reduced level of gene function of at least one gene encoding a protein that metabolizes an inducer of at least one inducible promoter; (c) a reduced level of gene function of at least one gene encoding a protein involved in biosynthesis of an inducer of at least one inducible promoter; (d) an altered gene function of a gene that affects the reduction/oxidation environment of the host cell cytoplasm; (e) a reduced level of gene function of a gene that encodes a reductase; (f) at least one expression construct encoding at least one disulfide bond isomerase protein; (g) at least one polynucleotide encoding a form of DsbC lacking a signal peptide; and/or (h) at least one polynucleotide encoding Ervlp.

In still other embodiments, an aforementioned method is provided wherein the expression construct is an extrachromosomal construct selected from the group consisting of a polynucleotide, a plasmid, and an artificial chromosome. In one embodiment, the expression construct comprises an inducible promoter. In another embodiment, the expression construct comprises two or more inducible promoters. In still another embodiment, at least one inducible promoter is a propionate-inducible promoter and at least one other inducible promoter is an L-arabinose-inducible promoter.

In yet other embodiments, an aforementioned method is provided wherein the growth substrate is selected from the group consisting of a selective media. In some embodiments, an aforementioned method is provided wherein the growth substrate comprises a matrix of 2 or more synergistic selection agents. In one embodiment, the 2 synergistic selection agents are trimethoprim and sulfamethoxazole, and the agents are present in the growth media at a concentration range of 1 ug/ml to 1000 ug/ml and 1 ug/ml to 1000 ug/ml, respectively.

In some embodiments, an aforementioned method is provided wherein the conditions that allow expression of the fusion protein include the presence of one or more inducers of expression of the fusion protein. In some embodiments, an aforementioned method is provided wherein the host cells are identified using a technique selected from the group consisting of visual inspection, chemiluminescence, radiography, fluorescence and colorimetric analyses. In still another embodiment, an aforementioned method is provided wherein the visualizing host cells in step (c) comprises detecting growth on agar plates.

In some embodiments, the present disclosure provides an aforementioned method further comprising a solid phase assay to detect soluble protein expression. For example, in one embodiment, an aforementioned method is provided further comprising the steps of: (a) plating the host cells on a growth substrate and incubating the host cells under conditions that allow host cell growth on the growth substrate; (b) optionally preparing at least one replica plate and incubating said replica plate under conditions that allow host cell growth and production of the protein of interest; (c) transferring host cells from the growth substrate or the at the least one replica plate of (b) to a membrane; (d) preparing the host cells that have been transferred to the membrane in (c) for probing, comprising (i) optionally fixing the host cells under conditions that allow immobilization of cellular components; (ii) blocking the host cells; and (iii) optionally lysing the host cells under conditions that allow permeabilization of the host cells; (e) contacting the permeabilized host cells with a probe solution comprising at least one probe under conditions that allow binding of the at least one probe the protein of interest, and thereby forming a probe-protein of interest complex; and (f) imaging the host cells under conditions that allow identifying the host cell that produces the protein of interest.

Additional embodiments are provided below, wherein the protein of interest is encoded by a gene product of interest. In one embodiment, the present disclosure provides a method of identifying a host cell that produces a gene product of interest, said method comprising the steps of: (a) plating a population of host cells on a growth substrate, wherein said host cells comprise an expression construct encoding one or more gene products, and incubating the host cells under conditions that allow host cell growth on the growth substrate; (b) preparing at least one replica plate and incubating said replica plate under conditions that allow host cell growth and production of the gene product; (c) transferring host cells from the at the least one replica plate of (b) to a membrane; (d) preparing the host cells that have been transferred to the membrane in (c) for probing, comprising (i) optionally fixing the host cells under conditions that allow immobilization of cellular components; (ii) blocking the host cells; and (iii) optionally lysing the host cells under conditions that allow permeabilization of the host cells; (e) contacting the permeabilized host cells with a probe solution comprising at least one probe under conditions that allow binding of the at least one probe to a gene product of interest, and thereby forming a probe-gene product complex; and (f) imaging the host cells under conditions that allow identifying the host cell that produces the gene product of interest. In another embodiment, step (c) further comprises marking the at least one replica plate and membrane to allow spatial alignment of the at least one replica plate and membrane.

In still other embodiments, an aforementioned method is provided wherein the host cell is selected from the group consisting of a eukaryotic cell, a prokaryotic cell, a bacterial cell, a mammalian cell and an insect cell. In still other embodiments, an aforementioned method is provided wherein the gene product of interest is selected from the group consisting of a therapeutic protein, an antibody, a Fab, a scFv, a nanobody, a T cell receptor, and a chimeric antigen receptor, or fragments thereof. In one embodiment, the gene product of interest is an intracellular protein.

In yet other embodiments, an aforementioned method is provided wherein the population of host cells comprises approximately one thousand to approximately one billion host cells. In other embodiments, the population of host cells have been modified to produce a gene product from an expression construct. In one embodiment, the expression construct comprises two or more inducible promoters. In still another embodiment, at least one inducible promoter is a propionate-inducible promoter and at least one other inducible promoter is an L-arabinose-inducible promoter.

The present disclosure also provides, in some embodiments, an aforementioned method wherein the host cells have been genetically modified to comprise one or more of: (a) an alteration of gene function of at least one gene encoding a transporter protein for an inducer of at least one inducible promoter; (b) a reduced level of gene function of at least one gene encoding a protein that metabolizes an inducer of at least one inducible promoter; (c) a reduced level of gene function of at least one gene encoding a protein involved in biosynthesis of an inducer of at least one inducible promoter; (d) an altered gene function of a gene that affects the reduction/oxidation environment of the host cell cytoplasm; (e) a reduced level of gene function of a gene that encodes a reductase; (f) at least one expression construct encoding at least one disulfide bond isomerase protein; (g) at least one polynucleotide encoding a form of DsbC lacking a signal peptide; and/or (h) at least one polynucleotide encoding Ervlp.

In still other embodiments, an aforementioned method is provided wherein 2, 3, 4, 5 or more replica plates are prepared. In one embodiment, the replica plates contain an antibiotic and at least one inducers of expression of the gene product of interest. In still other embodiments, the membrane is selected from the group consisting of a nitrocellulose membrane and a PVDF (polyvinylidene fluoride) membrane. In other embodiments, the replica plate and membrane of step (c) are contacted with a colored substance. In one embodiment, the colored substance is selected from the group consisting of acrylic paint and a dye. In another embodiment, the colored substance is contacted to the plate and membrane with an instrument selected from the group consisting of a needle and a pen.

In other embodiments, an aforementioned method is provided wherein the conditions that allow immobilization of cellular components comprise contacting the membrane with a composition comprising one or more of glutaraldehyde and paraformaldehyde. In other embodiments, the conditions that allow permeabilization of the host cells comprise contacting the membrane with a composition comprising one or more of lysozyme and EDTA.

In still other embodiments, an aforementioned method is provided wherein the at least one probe is selected from the group consisting of an antibody or functional fragment thereof, a nucleic acid-binding protein or functional fragment thereof, a receptor or functional fragment thereof, a ligand or functional fragment thereof, and antigen or functional fragment thereof, and a peptide or polypeptide capable of being bound by the gene product of interest. In one embodiment, the probe comprises a reporter moiety selected from the group consisting of biotin, a histidine tag, a Fc tag, a spy tag, a Strp tag, and an Avi tag.

The present disclosure also provides, in some embodiments, an aforementioned method wherein, when 2, 3, 4, 5 or more replica plates have been prepared, each membrane prepared from the replica plates is contacted with a different concentration of the probe solution. In still other embodiments, an aforementioned method is provided wherein the imaging comprises contacting the membrane with a composition comprising an activator of the reporter moiety. In another embodiment, the contacting the membrane with a composition comprising an activator of the reporter moiety is repeated once, twice, or three or more times. In still another embodiment, the activator is selected from the group consisting of alkaline phosphatase, streptavidin alkaline phosphatase, and streptavidin alkaline phosphatase dextran polymer.

In still other embodiments, an aforementioned method is provided wherein the imaging comprises a method selected from the group consisting of chemiluminescence, radiography, fluorescence and colorimetric analyses. In still other embodiments, an aforementioned is provided further comprising the step of re-plating one or more host cells that have been identified as capable of producing the gene product of interest. In another embodiment, re-plated host cell is subjected to the method of claim 1 to confirm the host cell's capability to produce the gene product of interest and/or to isolate the host cell strain from other host cell strains that have been identified as capable of producing the gene product of interest. In some embodiments the methods may further comprise determining the affinity of the probe-gene product complex.

The present disclosure also provides, in one embodiment, a method of screening a host cell from a population of host cells that produces a gene product of interest, said method comprising the steps of: (a) plating a population of host cells on a growth substrate, wherein said host cells comprise an expression construct encoding one or more gene products, and incubating the host cells under conditions that allow host cell growth on the growth substrate; (b) preparing at least one replica plate and incubating said replica plate under conditions that allow host cell growth and production of the gene product; (c) transferring host cells from the at the least one replica plate of (b) to a membrane; (d) preparing the host cells that have been transferred to the membrane in (c) for probing, comprising (i) fixing the host cells under conditions that allow immobilization of cellular components; (ii) blocking the host cells; and (iii) optionally lysing the host cells under conditions that allow permeabilization of the host cells; (e) contacting the permeabilized host cells with a probe solution comprising at least one probe under conditions that allow binding of the at least one probe to a gene product of interest, and thereby forming a probe-gene product complex; and (f) imaging the host cells under conditions that allow identifying the host cell that produces the gene product of interest.

In still another embodiment, the present disclosure provides a method of determining the relative affinity of a probe-gene product of interest, said method comprising the steps of: (a) plating a population of host cells on a growth substrate, wherein said host cells comprise an expression construct encoding one or more gene products, and incubating the host cells under conditions that allow host cell growth on the growth substrate; (b) preparing at least three replica plates and incubating said replica plate under conditions that allow host cell growth and production of the gene product; (c) transferring host cells from the at the least three replica plates of (b) to separate membranes; (d) preparing the host cells that have been transferred to the membrane in (c) for probing, comprising (i) fixing the host cells under conditions that allow immobilization of cellular components; (ii) blocking the host cells; and (iii) optionally lysing the host cells under conditions that allow permeabilization of the host cells; (e) contacting the permeabilized host cells on the at least three replica membranes with a probe solution comprising at least one probe under conditions that allow binding of the at least one probe to a gene product of interest, and thereby forming a probe-gene product complex, and wherein the probe solution comprises a different probe concentration; and (f) imaging the host cells under conditions that allow determining the relative affinity of a probe-gene product of interest.

### BRIEF DESCRIPTION OF THE DRAWINGS

FIG. 1 shows that cells harboring a mCherry-FolA fusion are highly resistant to trimethoprim.

FIG. 2 shows that synergy with sulfamethoxazole improves trimethoprim dynamic range.

FIG. 3 shows a matrix of trimethoprim-sulfamethoxazole.

FIG. 4 shows an exemplary plasmid construct.

FIG. 5 shows plates with colonies after 1 week of incubation.

FIG. 6 shows images from results of round 1 of a solid phase assay.

FIG. 7 shows round 2 results.

FIG. 8 shows an image of a processed membrane.

FIG. 9 shows an exemplary plasmid construct.

FIG. 10 shows additional results of a solid phase assay.

FIG. 11 shows results of liquid performers compared to agar plates.

### DETAILED DESCRIPTION

Embodiments of the present disclosure provide compositions and methods for identifying cells or strains from a library of cells or strains that can produce high amounts of a soluble protein of interest. In some embodiment, the protein of interest is genetically fused to a selection protein, such as antibiotic resistance gene or a protein that is a target of an antibiotic. As described herein, in some embodiments the selection process includes using 2 (or more) selection agents that act synergistically to improve the dynamic range of the assay.

Considerations for the fusion partner (i.e., selection protein) to enable selection include, but are not limited to: (1) Enables growth/no growth selection, (2) Enables the identification of a gradation of expression strains, from low to very high, by modifying the selection conditions and (3) Clean (low background noise). A selectable assay should allow testing of large diversity libraries into the billions of variants on a single 10 cm agar plate.

As described herein, each 10 cm plate may comprise up to 10 billion cells (e.g., 10 billion for cidal combinations of agents, 1 billion for static combinations agents).

### Identifying Host Cells Capable of Producing Soluble Proteins of Interest

In one embodiment of the present disclosure, a growth/no growth selection strategy is provided to identify E. coli strains in a mixed population (library) that produce higher soluble protein expression (i.e., relative to other strains in the library). As described herein, synergy of the selection agents increases the dynamic range of the assay. By way of example, a protein of interest is genetically engineered and fused to a reporter gene (folA) that is the target of an antibiotic (trimethoprim). In this exemplary embodiment, the choice of the reporter gene (folA)/antibiotic (trimethoprim) pair permits synergy with a second antibiotic agent to improve the potency of the primary antibiotic (trimethoprim). The benefits of a successful growth/no growth selection strategy thus permits the testing of several high complexity (>109) libraries in parallel.

The fusion proteins described herein can be to any antibiotic target or antibiotic resistance gene as long as a second, synergistic agent can be used to increase the useful dynamic range of the assay.

### Fusion Proteins

### Proteins of Interest

As described herein, a protein of interest (and, in some embodiments described herein, a gene of interest encoding the protein of interest) can be used with the methods provided herein. As used herein, a “soluble protein” or “soluble protein of interest” refers in one embodiment to a protein that will remain stable in solution and does not sediment over time or will remain in solution when a centrifugal force (16,000×g) is applied for ≥10 min. Protein solubility is a thermodynamic parameter defined, in some embodiments, as the concentration of protein in a saturated solution that is in equilibrium with a solid phase, either crystalline or amorphous, under a given set of conditions. Solubility can be influenced by a number of extrinsic and intrinsic factors including pH, ionic strength, temperature, and the presence of various solvent additives (Kramer, R. M., et al., Biphys J., 2012, 102 (8): 1907-1915). In one embodiment, soluble proteins are those with a solubility of more than 70% and insoluble with a solubility of less than 30% (Chan, P., et al., Scientific Reports, 2013, 3, 3333).

Proteins may include biologically active derivatives or variants or fragments. As used herein “biologically active derivative” or “biologically active variant” includes any derivative or variant of a molecule having substantially the same functional and/or biological properties of said molecule, such as binding properties, and/or the same structural basis, such as a peptidic backbone or a basic polymeric unit.

An “analog,” such as a “variant” or a “derivative,” is a compound substantially similar in structure and having the same biological activity, albeit in certain instances to a differing degree, to a naturally-occurring molecule. For example, a polypeptide variant refers to a polypeptide sharing substantially similar structure and having the same biological activity as a reference polypeptide. Variants or analogs differ in the composition of their amino acid sequences compared to the naturally-occurring polypeptide from which the analog is derived, based on one or more mutations involving (i) deletion of one or more amino acid residues at one or more termini of the polypeptide and/or one or more internal regions of the naturally-occurring polypeptide sequence (e.g., fragments), (ii) insertion or addition of one or more amino acids at one or more termini (typically an “addition” or “fusion”) of the polypeptide and/or one or more internal regions (typically an “insertion”) of the naturally-occurring polypeptide sequence or (iii) substitution of one or more amino acids for other amino acids in the naturally-occurring polypeptide sequence. By way of example, a “derivative” is a type of analog and refers to a polypeptide sharing the same or substantially similar structure as a reference polypeptide that has been modified, e.g., chemically.

A variant polypeptide is a type of analog polypeptide and includes insertion variants, wherein one or more amino acid residues are added to a biomolecule amino acid sequence of the disclosure. Insertions may be located at either or both termini of the protein, and/or may be positioned within internal regions of the therapeutic protein amino acid sequence. Insertion variants, with additional residues at either or both termini, include for example, fusion proteins and proteins including amino acid tags or other amino acid labels. In one aspect, the biomolecule optionally contains an N-terminal Met, especially when the molecule is expressed recombinantly in a bacterial cell such as E. coli. In another aspect, the biomolecule includes histidine tag (His-tag).

In deletion variants, one or more amino acid residues in a biomolecule polypeptide as described herein are removed. Deletions can be effected at one or both termini of the protein polypeptide, and/or with removal of one or more residues within the therapeutic protein amino acid sequence. Deletion variants, therefore, include fragments of a protein polypeptide sequence.

In substitution variants, one or more amino acid residues of a biomolecule are removed and replaced with alternative residues. In one aspect, the substitutions are conservative in nature and conservative substitutions of this type are well known in the art. Alternatively, the disclosure embraces substitutions that are also non-conservative. Exemplary conservative substitutions are described in Lehninger, [Biochemistry, 2nd Edition; Worth Publishers, Inc., New York (1975), pp. 71-77] and are set out immediately below.

Proteins contemplated herein include full-length proteins, precursors of full-length proteins, biologically active subunits or fragments of full length proteins, as well as biologically active derivatives and variants of any of these forms of therapeutic proteins. Thus, proteins include those that (1) have an amino acid sequence that has greater than about 60%, about 65%, about 70%, about 75%, about 80%, about 85%, about 90%, about 91%, about 92%, about 93%, about 94%, about 95%, about 96%, about 97%, about 98% or about 99% or greater amino acid sequence identity, over a region of at least about 25, about 50, about 100, about 200, about 300, about 400, or more amino acids, to a polypeptide encoded by a referenced nucleic acid or an amino acid sequence described herein. According to the present disclosure, the term “recombinant protein” includes any protein obtained via recombinant DNA technology. In certain embodiments, the term encompasses proteins as described herein.

In some embodiments, the protein is a therapeutic protein such as monoclonal or polyclonal antibody or other glycoprotein, a biosimilar, a Fc-fusion, an enzyme, a vaccine, a hormone, a cytokine, or a growth factor. In still other embodiments, gene product is an anticoagulant, a blood factor, a bone morphogenic protein, an interleukin, an interferon, a thrombolytic, or any protein produced by recombinant means. In other embodiments, the methods provide that the methods can be used with any biomolecule molecule or chemical entity, including small molecules, that are produced by cells described herein and that have a probe that can be used for binding steps.

The term “antibody” as used herein refers to whole antibodies that interact with (e.g., by binding, steric hindrance, stabilizing/destabilizing, spatial distribution) an epitope on a target antigen. A naturally occurring “antibody” is a glycoprotein comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. The term “antibody” includes for example, monoclonal antibodies, human antibodies, humanized antibodies, camelised antibodies, chimeric antibodies, single-chain Fvs (scFv), disulfide-linked Fvs (sdFv), Fab fragments, F (ab′) fragments, and anti-idiotypic (anti-Id) antibodies (including, e.g., anti-Id antibodies to antibodies of the invention), and epitope-binding fragments of any of the above. The antibodies can be of any isotype (e.g., IgG, IgE, IgM, IgD, IgA and IgY), class (e.g., IgG1, IgG2, IgG3, IgG4, IgA1 and IgA2) or subclass. The antibody or epitope-binding fragments may be, or be a component of, a multi-specific molecule, including a bi-specific antibody.

Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention the numbering of the constant region domains increases as they become more distal from the antigen binding site or amino-terminus of the antibody. The N-terminus is a variable region and at the C-terminus is a constant region; the CH3 and CL domains actually comprise the carboxy-terminus of the heavy and light chain, respectively.

The phrase “antibody fragment”, as used herein, refers to one or more portions of an antibody that retain the ability to specifically interact with (e.g., by binding, steric hindrance, stabilizing/destabilizing, spatial distribution) a target epitope. Examples of binding fragments include, but are not limited to, a Fab fragment, a monovalent fragment consisting of the VL, VH, CL and CH1 domains; a F (ab) 2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., (1989) Nature 341:544-546), which consists of a VH domain; and an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al., (1988) Science 242:423-426; and Huston et al., (1988) Proc. Natl. Acad. Sci. 85:5879-5883). Such single chain antibodies are also intended to be encompassed within the term “antibody fragment”. These antibody fragments are obtained using conventional techniques known to those of skill in the art, and the fragments are screened for utility in the same manner as are intact antibodies.

In other embodiments, the protein of interest is a therapeutic protein, a T cell receptor, and a chimeric antigen receptor, a growth factor, a cytokine, a hormone, an enzyme, or a functional fragment thereof. In one embodiment, the protein is insulin.

The present disclosure also provides, in some embodiments, a fusion protein that includes an optional linker, e.g., a flexible linker sequence, between two protein components. In some embodiments, the linker is at least 5, at least 10, at least 15, at least 20, at least 25, at least 30, at least 35, at least 40, at least 45, or at least 50 amino acids in length. In other embodiments, the linker is less than 5, less than 10, less than 15, less than 20, less than 25, less than 30, less than 35, less than 40, less than 45, or less than 50 amino acids in length. In one embodiment, the linker is 23 amino acids in length. The linkers may be, in various embodiments, flexible, rigid, or cleavable (e.g., disulfide, protease sensitive sequences) (Chen, X., et al., Advanced Drug Delivery Reviews, 65 (1): 1357-1369 (2013)). Linkers may be derived from naturally occurring multi-domain proteins, or they may be empirical (See Argos, P., J. Mol. Biol., 211:943-958 (1990); and Heringa, G. R., Protein Eng., 15:871-879 (2002) which independently compared several properties of natural linkers, such as length, hydrophobicity, amino acid residues, and secondary structure). Threonine (Thr), serine (Ser), proline (Pro), glycine (Gly), aspartic acid (Asp), lysine (Lys), glutamine (Gln), asparagine (Asn), and alanine (Ala) are, in some embodiments, preferable linker constituents, as are arginine (Arg), phenylalanine (Phe), and glutamic acid (Glu). In general, preferable amino acids are polar uncharged or charged residues. Natural linkers adopt various secondary structures, such as helical, β-strand, coil/bend and turns, to exert their functions.

### Selection Proteins and Selection Agents

The selection proteins provided herein are, in some embodiments, proteins, polypeptides, or analogs such as variants or derivatives described above. Any selection agents can be used in the methods provided by the present disclosure, so long as the agents work synergistically to improve the dynamic range. “Synergistic selection agents” refers to any combination of two or more agents where the effect of the combination of agents is greater than the sum of the individual agents alone. Synergistic selection agents include, but are not limited to, antibiotics, sugars, chemical agents, enzyme substrate and analogs, enzyme inhibitors, agents that sequester biomolecules, chelating agents, as well as agents that compromise the cell wall or cell membrane. The selection agents can be part of the composition that makes up the growth substrate, or they can be provided separately.

In one example, the selection protein is dihydrofolate reductase (dhfr) and the synergistic selection agents are trimethoprim and sulfamethoxazole. In certain embodiments, the dhfr is from a bacterial source. Typically, bacterial dhfr are designated FolA. In preferred embodiments, the FolA is derived from E. coli. In one embodiment, the endogenous FolA is knocked-out or otherwise rendered inactive in the cell or strain that is used according to the methods described herein. In one embodiment, the FolA is derived from E. coli as follows (www.uniprot.org/uniprot/P0ABQ4.fasta):

| | |
|---|---|
| SEQ ID NO: 1 | MISLIAALAVDRVIGMENAMPWNLPADLAWFKRNT |
| FolA | LNKPVIMGRHTWESIGRPLPGRKNIILSSQPGTDD |
| | RVTWVKSVDEAIAACGDVPEIMVIGGGRVYEQFLP |
| | KAQKLYLTHIDAEVEGDTHFPDYEPDDWESVFSEF |
| | HDADAQNSHSYCFEILERR |

In other embodiments, the selection protein and synergistic selection agents include, but are not limited to, synergistic combinations of trimethoprim/sulfamethoxazole, b-lactam/b-lactamase inhibitor, cell wall active agents/aminoglycosides, and fosfommycin/b-lactams.

### Cells

Cells, including cells from a population of cells or cells that are part of a library of cells are contemplated by the present disclosure. As used herein, a “library” with respect to cells refers to any collection of genetically distinct variants of cells that are either intentionally engineered or naturally occurring. For example, cells with unique genotypes and/or that are uniquely genetically engineered are contemplated. Exemplary genetic modifications are described herein, including modifications that allow the cell to express and produce a recombinant, heterologous protein of interest. Unique genotypes or cells that have been uniquely engineered refers, in some embodiments, to a population of cells that differ from one in another in a genetic aspect—e.g., the presence of a mutation, a plasmid, a gene within a plasmid, and the like.

Cells comprising one or more of the expression constructs described herein are contemplated in various embodiments of the present disclosure. Cells of the present disclosure include an outer membrane (e.g., comprised of protein and lipids) within which the fusion proteins described herein may interact or otherwise reside once they are expressed.

Prokaryotic host cells. In some embodiments of the disclosure, expression constructs designed for expression of gene products, including fusion proteins as described herein, are provided in host cells, such as prokaryotic host cells. Prokaryotic host cells can include archaea (such as Haloferax volcanii, Sulfolobus solfataricus), Gram-positive bacteria (such as Bacillus subtilis, Bacillus licheniformis, Brevibacillus choshinensis, Lactobacillus brevis, Lactobacillus buchneri, Lactococcus lactis, and Streptomyces lividans), or Gram-negative bacteria, including Alphaproteobacteria (Agrobacterium tumefaciens, Caulobacter crescentus, Rhodobacter sphaeroides, and Sinorhizobium meliloti), Betaproteobacteria (Alcaligenes eutrophus), and Gammaproteobacteria (Acinetobacter calcoaceticus, Azotobacter vinelandii, Escherichia coli, Pseudomonas aeruginosa, and Pseudomonas putida). Preferred host cells include Gammaproteobacteria of the family Enterobacteriaceae, such as Enterobacter, Erwinia, Escherichia (including E. coli), Klebsiella, Proteus, Salmonella (including Salmonella typhimurium), Serratia (including Serratia marcescans), and Shigella.

Eukaryotic host cells. Many additional types of host cells can be used for the expression systems of the present disclosure, including eukaryotic cells such as yeast (Candida shehatae, Kluyveromyces lactis, Kluyveromyces fragilis, other Kluyveromyces species, Pichia pastoris, Saccharomyces cerevisiae, Saccharomyces pastorianus also known as Saccharomyces carlsbergensis, Schizosaccharomyces pombe, Dekkera/Brettanomyces species, and Yarrowia lipolytica); other fungi (Aspergillus nidulans, Aspergillus niger, Neurospora crassa, Penicillium, Tolypocladium, Trichoderma reesia); insect cell lines (Drosophila melanogaster Schneider 2 cells and Spodoptera frugiperda Sf9 cells); and mammalian cell lines including immortalized cell lines (Chinese hamster ovary (CHO) cells, HeLa cells, baby hamster kidney (BHK) cells, monkey kidney cells (COS), human embryonic kidney (HEK, 293, or HEK-293) cells, and human hepatocellular carcinoma cells (Hep G2)). The above host cells are available from the American Type Culture Collection.

As described in WO/2017/106583, incorporated by reference in its entirety herein, producing gene products such as therapeutic proteins at commercial scale and in soluble form is addressed by providing suitable host cells capable of growth at high cell density in fermentation culture, and which can produce soluble gene products in the oxidizing host cell cytoplasm through highly controlled inducible gene expression. Host cells of the present disclosure with these qualities are produced by combining some or all of the following characteristics. (1) The host cells are genetically modified to have an oxidizing cytoplasm, through increasing the expression or function of oxidizing polypeptides in the cytoplasm, and/or by decreasing the expression or function of reducing polypeptides in the cytoplasm. Specific examples of such genetic alterations are provided herein. Optionally, host cells can also be genetically modified to express chaperones and/or cofactors that assist in the production of the desired gene product(s), and/or to glycosylate polypeptide gene products. (2) The host cells comprise one or more expression constructs designed for the expression of one or more gene products of interest; in certain embodiments, at least one expression construct comprises an inducible promoter and a polynucleotide encoding a gene product to be expressed from the inducible promoter. (3) The host cells contain additional genetic modifications designed to improve certain aspects of gene product expression from the expression construct(s). In particular embodiments, the host cells (A) have an alteration of gene function of at least one gene encoding a transporter protein for an inducer of at least one inducible promoter, and as another example, wherein the gene encoding the transporter protein is selected from the group consisting of araE, araE, araG, araH, rhaT, xylF, xylG, and xylH, or particularly is araE, or wherein the alteration of gene function more particularly is expression of araE from a constitutive promoter; and/or (B) have a reduced level of gene function of at least one gene encoding a protein that metabolizes an inducer of at least one inducible promoter, and as further examples, wherein the gene encoding a protein that metabolizes an inducer of at least one said inducible promoter is selected from the group consisting of araA, araB, araD, prpB, prpD, rhaA, rhaB, rhaD, xylA, and xylB; and/or (C) have a reduced level of gene function of at least one gene encoding a protein involved in biosynthesis of an inducer of at least one inducible promoter, which gene in further embodiments is selected from the group consisting of scpA/sbm, argK/ygfD, scpB/ygfG, scpC/ygfH, rmlA, rmlB, rmlC, and rmlD.

Host Cells with Oxidizing Cytoplasm. The expression systems of the present disclosure are designed to express gene products; in certain embodiments of the disclosure, the gene products are expressed in a host cell. Examples of host cells are provided that allow for the efficient and cost-effective expression of gene products, including components of multimeric products. Host cells can include, in addition to isolated cells in culture, cells that are part of a multicellular organism, or cells grown within a different organism or system of organisms. In certain embodiments of the disclosure, the host cells are microbial cells such as yeasts (Saccharomyces, Schizosaccharomyces, etc.) or bacterial cells, or are gram-positive bacteria or gram-negative bacteria, or are E. coli, or are an E. coli B strain, or are E. coli (B strain) EB0001 cells (also called E. coli ASE (DGH) cells), or are E. coli (B strain) EB0002 cells. In growth experiments with E. coli host cells having oxidizing cytoplasm, specifically the E. coli B strains SHuffle® Express (NEB Catalog No. C3028H) and SHuffle® T7 Express (NEB Catalog No. C3029H) and the E. coli K strain SHuffle® T7 (NEB Catalog No. C3026H), these E. coli B strains with oxidizing cytoplasm are able to grow to much higher cell densities than the most closely corresponding E. coli K strain (WO/2017/106583).

Alterations to host cell gene functions. Certain alterations can be made to the gene functions of host cells comprising inducible expression constructs, to promote efficient and homogeneous induction of the host cell population by an inducer. Preferably, the combination of expression constructs, host cell genotype, and induction conditions results in at least 75% (more preferably at least 85%, and most preferably, at least 95%) of the cells in the culture expressing gene product from each induced promoter, as measured by the method of Khlebnikov et al. described in Example 9 of WO/2017/106583. For host cells other than E. coli, these alterations can involve the function of genes that are structurally similar to an E. coli gene, or genes that carry out a function within the host cell similar to that of the E. coli gene. Alterations to host cell gene functions include eliminating or reducing gene function by deleting the gene protein-coding sequence in its entirety, or deleting a large enough portion of the gene, inserting sequence into the gene, or otherwise altering the gene sequence so that a reduced level of functional gene product is made from that gene. Alterations to host cell gene functions also include increasing gene function by, for example, altering the native promoter to create a stronger promoter that directs a higher level of transcription of the gene, or introducing a missense mutation into the protein-coding sequence that results in a more highly active gene product. Alterations to host cell gene functions include altering gene function in any way, including for example, altering a native inducible promoter to create a promoter that is constitutively activated. In addition to alterations in gene functions for the transport and metabolism of inducers, as described herein with relation to inducible promoters, and/or an altered expression of chaperone proteins, it is also possible to alter the reduction-oxidation environment of the host cell.

Host cell reduction-oxidation environment. In bacterial cells such as E. coli, proteins that need disulfide bonds are typically exported into the periplasm where disulfide bond formation and isomerization is catalyzed by the Dsb system, comprising DsbABCD and DsbG. Increased expression of the cysteine oxidase DsbA, the disulfide isomerase DsbC, or combinations of the Dsb proteins, which are all normally transported into the periplasm, has been utilized in the expression of heterologous proteins that require disulfide bonds (Makino et al., Microb Cell Fact 2011 May 14; 10:32). It is also possible to express cytoplasmic forms of these Dsb proteins, such as a cytoplasmic version of DsbA and/or of DsbC (‘cDsbA or ‘cDsbC’), that lacks a signal peptide and therefore is not transported into the periplasm. Cytoplasmic Dsb proteins such as cDsbA and/or cDsbC are useful for making the cytoplasm of the host cell more oxidizing and thus more conducive to the formation of disulfide bonds in heterologous proteins produced in the cytoplasm. The host cell cytoplasm can also be made less reducing and thus more oxidizing by altering the thioredoxin and the glutaredoxin/glutathione enzyme systems directly: mutant strains defective in glutathione reductase (gor) or glutathione synthetase (gshB), together with thioredoxin reductase (trxB), render the cytoplasm oxidizing. These strains are unable to reduce ribonucleotides and therefore cannot grow in the absence of exogenous reductant, such as dithiothreitol (DTT). Suppressor mutations (such as ahpC* and ahpCA, Lobstein et al., Microb Cell Fact 2012 May 8; 11:56; doi: 10.1186/1475-2859-11-56) in the gene ahpC, which encodes the peroxiredoxin AhpC, convert it to a disulfide reductase that generates reduced glutathione, allowing the channeling of electrons onto the enzyme ribonucleotide reductase and enabling the cells defective in gor and trxB, or defective in gshB and trxB, to grow in the absence of DTT. A different class of mutated forms of AhpC can allow strains, defective in the activity of gamma-glutamylcysteine synthetase (gshA) and defective in trxB, to grow in the absence of DTT; these include AhpC V164G, AhpC S71F, AhpC E173/S71F, AhpC E171Ter, and AhpC dupl62-169 (Faulkner et al., Proc Natl Acad Sci USA 2008 May 6; 105 (18): 6735-6740, Epub 2008 May 2). In such strains with oxidizing cytoplasm, exposed protein cysteines become readily oxidized in a process that is catalyzed by thioredoxins, in a reversal of their physiological function, resulting in the formation of disulfide bonds. Other proteins that may be helpful to reduce the oxidative stress effects in host cells of an oxidizing cytoplasm are HPI (hydroperoxidase I) catalase-peroxidase encoded by E. coli katG and HPII (hydroperoxidase II) catalase-peroxidase encoded by E. coli katE, which disproportionate peroxide into water and 02 (Farr and Kogoma, Microbiol Rev. 1991 December; 55 (4): 561-585; Review). Increasing levels of KatG and/or KatE protein in host cells through induced coexpression or through elevated levels of constitutive expression is an aspect of some embodiments of the disclosure.

Another alteration that can be made to host cells is to express the sulfhydryl oxidase Ervlp from the inner membrane space of yeast mitochondria in the host cell cytoplasm, which has been shown to increase the production of a variety of complex, disulfide-bonded proteins of eukaryotic origin in the cytoplasm of E. coli, even in the absence of mutations in gor or trxB (Nguyen et al, Microb Cell Fact 2011 Jan. 7; 10:1).

Host cells comprising expression constructs preferably also express cDsbA and/or cDsbC and/or Ervlp; are deficient in trxB gene function; are also deficient in the gene function of either gor, gshB, or gshA; optionally have increased levels of katG and/or katE gene function; and express an appropriate mutant form of AhpC so that the host cells can be grown in the absence of DTT.

Chaperones. In some embodiments, desired gene products are coexpressed with other gene products, such as chaperones, that are beneficial to the production of the desired gene product. Chaperones are proteins that assist the non-covalent folding or unfolding, and/or the assembly or disassembly, of other gene products, but do not occur in the resulting monomeric or multimeric gene product structures when the structures are performing their normal biological functions (having completed the processes of folding and/or assembly). Chaperones can be expressed from an inducible promoter or a constitutive promoter within an expression construct, or can be expressed from the host cell chromosome; preferably, expression of chaperone protein(s) in the host cell is at a sufficiently high level to produce coexpressed gene products that are properly folded and/or assembled into the desired product. Examples of chaperones present in E. coli host cells are the folding factors Dnak/DnaJ/GrpE, DsbC/DsbG, GroEL/GroES, IbpA/IbpB, Skp, Tig (trigger factor), and FkpA, which have been used to prevent protein aggregation of cytoplasmic or periplasmic proteins. DnaK/DnaJ/GrpE, GroEL/GroES, and ClpB can function synergistically in assisting protein folding and therefore expression of these chaperones in combinations has been shown to be beneficial for protein expression (Makino et al., Microb Cell Fact 2011 May 14; 10:32). When expressing eukaryotic proteins in prokaryotic host cells, a eukaryotic chaperone protein, such as protein disulfide isomerase (PDI) from the same or a related eukaryotic species, is in certain embodiments of the disclosure coexpressed or inducibly coexpressed with the desired gene product.

One chaperone that can be expressed in host cells is a protein disulfide isomerase from Humicola insolens, a soil hyphomycete (soft-rot fungus). An amino acid sequence of Humicola insolens PDI is shown as SEQ ID NO: 1 of WO/2017/106583; it lacks the signal peptide of the native protein so that it remains in the host cell cytoplasm. The nucleotide sequence encoding PDI was optimized for expression in E. coli; the expression construct for PDI is shown as SEQ ID NO: 2 of WO/2017/106583. SEQ ID NO: 2 contains a GCTAGC NheI restriction site at its 5′ end, an AGGAGG ribosome binding site at nucleotides 7 through 12, the PDI coding sequence at nucleotides 21 through 1478, and a GTCGAC Sail restriction site at its 3′ end. The nucleotide sequence of SEQ ID NO: 2 was designed to be inserted immediately downstream of a promoter, such as an inducible promoter. The NheI and Sail restriction sites in SEQ ID NO: 2 can be used to insert it into a vector multiple cloning site, such as that of the pSOL expression vector (SEQ ID NO: 3 of WO/2017/106583), described in published US patent application US2015353940A1, which is incorporated by reference in its entirety herein. Other PDI polypeptides can also be expressed in host cells, including PDI polypeptides from a variety of species (Saccharomyces cerevisiae (UniProtKB PI 7967), Homo sapiens (UniProtKB P07237), Mus musculus (UniProtKB P09103), Caenorhabditis elegans (UniProtKB Q 17770 and Q 17967), Arabidopsis thaliana (UniProtKB 048773, Q9X101, Q9S G3, Q9LJU2, Q9MAU6, Q94F09, and Q9T042), Aspergillus niger (UniProtKB Q12730) and also modified forms of such PDI polypeptides. In certain embodiments of the disclosure, a PDI polypeptide expressed in host cells of the disclosure shares at least 70%, or 80%, or 90%, or 95% amino acid sequence identity across at least 50% (or at least 60%, or at least 70%, or at least 80%, or at least 90%) of the length of SEQ ID NO: 1 of WO/2017/106583, where amino acid sequence identity is determined according to Example 10 of WO/2017/106583.

Cellular transport of cofactors. When using the expression systems of the disclosure to produce enzymes that require cofactors for function, it is helpful to use a host cell capable of synthesizing the cofactor from available precursors, or taking it up from the environment. Common cofactors include ATP, coenzyme A, flavin adenine dinucleotide (FAD), NAD+/NADH, and heme. Polynucleotides encoding cofactor transport polypeptides and/or cofactor synthesizing polypeptides can be introduced into host cells, and such polypeptides can be constitutively expressed, or inducibly coexpressed with the gene products to be produced by methods of the disclosure.

Glycosylation of polypeptide gene products. Host cells can have alterations in their ability to glycosylate polypeptides. For example, eukaryotic host cells can have eliminated or reduced gene function in glycosyltransferase and/or oligo-saccharyltransferase genes, impairing the normal eukaryotic glycosylation of polypeptides to form glycoproteins. Prokaryotic host cells such as E. coli, which do not normally glycosylate polypeptides, can be altered to express a set of eukaryotic and prokaryotic genes that provide a glycosylation function (DeLisa et al., WO2009089154A2, 2009 Jul. 16).

Available host cell strains with altered gene functions. To create preferred strains of host cells to be used in the expression systems and methods of the disclosure, it is useful to start with a strain that already comprises desired genetic alterations (Table A; WO/2017/106583).

TABLE A
Exemplary host cell strains

| Strain | Genotype | Source |
|---|---|---|
| E, coli TOP10 | F-mcrA Δ(mrr-hsdRMS-mcrBC) | Invitrogen Life Technologies |
| | Φ80lacZ ΔM15 Δlacx74 recA1 araD139 | Catalog Nos. C404-10, |
| | Δ(ara-leu)7697 galU galK rpsL (Str^{R}) | C4040-03, C4040-06, C4040- |
| | endA1 nupG λ | 50, and C4040-52 |
| E, coli Origami ™ | Δ(ara-leu)7697 Δlacx74 ΔphoA PvuII | Merck (EMD Millipore |
| 2 | phoR araD139 ahpC gale galK rpsL | Chemicals) Catalog No. |
| | F’[lac⁺ lacI^{q} pro] gor522::Tn10 trxB | 71344 |
| | (Str^{R}, Tet^{R}) | |
| E, coli SHuffle ® | fhuA2 [lon] ompT ahpC gal λatt::pNEB3- | New England Biolabs |
| Express | r1-cDsbc (Spec, lacI) ΔtrxB sulA11 | Catalog No. C3028H |
| | R(mcr-73::miniTn10-Tet^{S})2 [dcm] | |
| | R(zgb-210::Tn10-Tet^{S}) endA1 Δgor | |
| | Δ(mcrC-mrr)114::IS10 | |

### Expression Constructs

In some embodiments of the present disclosure, inducible promoters are contemplated for use with the expression constructs. Exemplary promoters are described herein and are also described in WO/2016/205570, incorporated by reference in its entirety herein. As described herein, the cells comprising one or more expression constructs may optionally include one or more inducible promoters to express a gene product of interest. In one embodiment, the gene product is a fusion protein as described herein. In other embodiments, the gene product is a protein, for example therapeutic protein.

Expression Constructs. Expression constructs are polynucleotides designed for the expression of one or more gene products of interest, and thus are not naturally occurring molecules. Expression constructs can be integrated into a host cell chromosome, or maintained within the host cell as polynucleotide molecules replicating independently of the host cell chromosome, such as plasmids or artificial chromosomes. An example of an expression construct is a polynucleotide resulting from the insertion of one or more polynucleotide sequences into a host cell chromosome, where the inserted polynucleotide sequences alter the expression of chromosomal coding sequences. An expression vector is a plasmid expression construct specifically used for the expression of one or more gene products. One or more expression constructs can be integrated into a host cell chromosome or be maintained on an extrachromosomal polynucleotide such as a plasmid or artificial chromosome. The following are descriptions of particular types of polynucleotide sequences that can be used in expression constructs for the expression or coexpression of gene products, including fusion proteins as described herein.

Origins of replication. Expression constructs must comprise an origin of replication, also called a replicon, in order to be maintained within the host cell as independently replicating polynucleotides. Different replicons that use the same mechanism for replication cannot be maintained together in a single host cell through repeated cell divisions. As a result, plasmids can be categorized into incompatibility groups depending on the origin of replication that they contain, as shown in Table 2 of WO/2016/205570. Origins of replication can be selected for use in expression constructs on the basis of incompatibility group, copy number, and/or host range, among other criteria. As described above, if two or more different expression constructs are to be used in the same host cell for the coexpression of multiple gene products, it is best if the different expression constructs contain origins of replication from different incompatibility groups: a pMBl replicon in one expression construct and a pl5A replicon in another, for example. The average number of copies of an expression construct in the cell, relative to the number of host chromosome molecules, is determined by the origin of replication contained in that expression construct. Copy number can range from a few copies per cell to several hundred (Table 2 of WO/2016/205570). In one embodiment of the disclosure, different expression constructs are used which comprise inducible promoters that are activated by the same inducer, but which have different origins of replication. By selecting origins of replication that maintain each different expression construct at a certain approximate copy number in the cell, it is possible to adjust the levels of overall production of a gene product expressed from one expression construct, relative to another gene product expressed from a different expression construct. As an example, to coexpress subunits A and B of a multimeric protein, an expression construct is created which comprises the colEl replicon, the am promoter, and a coding sequence for subunit A expressed from the am promoter: ‘colEl-Para-A.

Another expression construct is created comprising the pl 5A replicon, the am promoter, and a coding sequence for subunit B: ‘pl5A-Para-B’. These two expression constructs can be maintained together in the same host cells, and expression of both subunits A and B is induced by the addition of one inducer, arabinose, to the growth medium. If the expression level of subunit A needed to be significantly increased relative to the expression level of subunit B, in order to bring the stoichiometric ratio of the expressed amounts of the two subunits closer to a desired ratio, for example, a new expression construct for subunit A could be created, having a modified pMB 1 replicon as is found in the origin of replication of the pUC9 plasmid (‘pUC9ori’): pUC9ori-Para-A. Expressing subunit A from a high-copy-number expression construct such as pUC9ori-Para-A should increase the amount of subunit A produced relative to expression of subunit B from pl5A-Para-B. In a similar fashion, use of an origin of replication that maintains expression constructs at a lower copy number, such as pSOOl (WO/2016/205570), could reduce the overall level of a gene product expressed from that construct. Selection of an origin of replication can also determine which host cells can maintain an expression construct comprising that replicon. For example, expression constructs comprising the colEl origin of replication have a relatively narrow range of available hosts, species within the Enterobacteriaceae family, while expression constructs comprising the RK2 replicon can be maintained in E. coli, Pseudomonas aeruginosa, Pseudomonas putida, Azotobacter vinelandii, and Alcaligenes eutrophus, and if an expression construct comprises the RK2 replicon and some regulator genes from the RK2 plasmid, it can be maintained in host cells as diverse as Sinorhizobium meliloti, Agrobacterium tumefaciens, Caulobacter crescentus, Acinetobacter calcoaceticus, and Rhodobacter sphaeroides (Kiies and Stahl, Microbiol Rev 1989 December; 53 (4): 491-516).

Similar considerations can be employed to create expression constructs for inducible expression or coexpression in eukaryotic cells. For example, the 2-micron circle plasmid of Saccharomyces cerevisiae is compatible with plasmids from other yeast strains, such as pSRl (ATCC Deposit Nos. 48233 and 66069; Araki et al., J Mol Biol 1985 Mar. 20; 182 (2): 191-203) and pKDl (ATCC Deposit No. 37519; Chen et al, Nucleic Acids Res 1986 Jun. 11; 14 (11): 4471-4481).

Selectable markers. Expression constructs usually comprise a selection gene, also termed a selectable marker, which encodes a protein necessary for the survival or growth of host cells in a selective culture medium. Host cells not containing the expression construct comprising the selection gene will not survive in the culture medium. Typical selection genes encode proteins that confer resistance to antibiotics or other toxins, or that complement auxotrophic deficiencies of the host cell. One example of a selection scheme utilizes a drug such as an antibiotic to arrest growth of a host cell. Those cells that contain an expression construct comprising the selectable marker produce a protein conferring drug resistance and survive the selection regimen. Some examples of antibiotics that are commonly used for the selection of selectable markers (and abbreviations indicating genes that provide antibiotic resistance phenotypes) are: ampicillin (AmpR), chloramphenicol (CmlR or CmR), kanamycin (KanR), spectinomycin (SpcR), streptomycin (StrR), and tetracycline (TetR). Many of the plasmids in Table 2 of WO/2016/205570 comprise selectable markers, such as pBR322 (AmpR, TetR); pMOB45 (CmR, TetR); pACYCIW (AmpR, KanR); and pGBMI (SpcR, StrR). The native promoter region for a selection gene is usually included, along with the coding sequence for its gene product, as part of a selectable marker portion of an expression construct. Alternatively, the coding sequence for the selection gene can be expressed from a constitutive promoter.

In various aspects, suitable selectable markers include, but are not limited to, neomycin phosphotransferase (npt II), hygromycin phosphotransferase (hpt), dihydrofolate reductase (dhfr), zeocin, phleomycin, bleomycin resistance gene (ble), gentamycin acetyltransferase, streptomycin phosphotransferase, mutant form of acetolactate synthase (als), bromoxynil nitrilase, phosphinothricin acetyl transferase (bar), enolpyruvylshikimate-3-phosphate (EPSP) synthase (aro A), muscle specific tyrosine kinase receptor molecule (MuSK-R), copper-zinc superoxide dismutase (sod1), metallothioneins (cup1, MT1), beta-lactamase (BLA), puromycin N-acetyl-transferase (pac), blasticidin acetyl transferase (bls), blasticidin deaminase (bsr), histidinol dehydrogenase (HDH), N-succinyl-5-aminoimidazole-4-carboxamide ribotide (SAICAR) synthetase (ade1), argininosuccinate lyase (arg4), beta-isopropylmalate dehydrogenase (leu2), invertase (suc2), orotidine-5′-phosphate (OMP) decarboxylase (ura3), and orthologs of any of the foregoing.

Inducible promoter. As described herein, there are several different inducible promoters that can be included in expression constructs as part of the inducible coexpression systems of the disclosure. Preferred inducible promoters share at least 80% polynucleotide sequence identity (more preferably, at least 90% identity, and most preferably, at least 95% identity) to at least 30 (more preferably, at least 40, and most preferably, at least 50) contiguous bases of a promoter polynucleotide sequence as defined in Table 1 of WO/2016/205570 by reference to the E. coli K-12 substrain MG1655 genomic sequence, where percent polynucleotide sequence identity is determined using the methods of Example 11 of WO/2016/205570. Under ‘standard’ inducing conditions (see Example 5 of WO/2016/205570), preferred inducible promoters have at least 75% (more preferably, at least 100%, and most preferably, at least 110%) of the strength of the corresponding ‘wild-type’ inducible promoter of E. coli K-12 substrain MG1655, as determined using the quantitative PCR method of De Mey et al. (Example 6 of WO/2016/205570). Within the expression construct, an inducible promoter is placed 5′ to (or ‘upstream of) the coding sequence for the gene product that is to be inducibly expressed, so that the presence of the inducible promoter will direct transcription of the gene product coding sequence in a 5′ to 3′ direction relative to the coding strand of the polynucleotide encoding the gene product.

Ribosome binding site. For polypeptide gene products, the nucleotide sequence of the region between the transcription initiation site and the initiation codon of the coding sequence of the gene product that is to be inducibly expressed corresponds to the 5′ untranslated region (‘UTR’) of the mRNA for the polypeptide gene product. Preferably, the region of the expression construct that corresponds to the 5′ UT comprises a polynucleotide sequence similar to the consensus ribosome binding site (RBS, also called the Shine-Dalgarno sequence) that is found in the species of the host cell. In prokaryotes (archaea and bacteria), the RBS consensus sequence is GGAGG or GGAGGU, and in bacteria such as E. coli, the RBS consensus sequence is AGGAGG or AGGAGGU. The RBS is typically separated from the initiation codon by 5 to 10 intervening nucleotides. In expression constructs, the RBS sequence is preferably at least 55% identical to the AGGAGGU consensus sequence, more preferably at least 70% identical, and most preferably at least 85% identical, and is separated from the initiation codon by 5 to 10 intervening nucleotides, more preferably by 6 to 9 intervening nucleotides, and most preferably by 6 or 7 intervening nucleotides. The ability of a given RBS to produce a desirable translation initiation rate can be calculated at the website salis.psu.edu/software/RBSLibraryCalculatorSearchMode, using the RBS Calculator; the same tool can be used to optimize a synthetic RBS for a translation rate across a 100,000+ fold range (Salis, Methods Enzymol 2011; 498:19-42).

Multiple cloning site. A multiple cloning site (MCS), also called a polylinker, is a polynucleotide that contains multiple restriction sites in close proximity to or overlapping each other. The restriction sites in the MCS typically occur once within the MCS sequence, and preferably do not occur within the rest of the plasmid or other polynucleotide construct, allowing restriction enzymes to cut the plasmid or other polynucleotide construct only within the MCS. Examples of MCS sequences are those in the pBAD series of expression vectors, including pBAD18, pBAD18-Cm, pBAD18-Kan, pBAD24, pBAD28, pBAD30, and pBAD33 (Guzman et al., J Bacteriol 1995 July; 177 (14): 4121-4130); or those in the pPRO series of expression vectors derived from the pBAD vectors, such as pPR018, pPR018-Cm, pPR018-Kan, pPR024, pPRO30, and pPR033 (U.S. Pat. No. 8,178,338 B2; May 15, 2012; Keasling, Jay). A multiple cloning site can be used in the creation of an expression construct: by placing a multiple cloning site 3′ to (or downstream of) a promoter sequence, the MCS can be used to insert the coding sequence for a gene product to be expressed or coexpressed into the construct, in the proper location relative to the promoter so that transcription of the coding sequence will occur. Depending on which restriction enzymes are used to cut within the MCS, there may be some part of the MCS sequence remaining within the expression construct after the coding sequence or other polynucleotide sequence is inserted into the expression construct. Any remaining MCS sequence can be upstream or, or downstream of, or on both sides of the inserted sequence. A ribosome binding site can be placed upstream of the MCS, preferably immediately adjacent to or separated from the MCS by only a few nucleotides, in which case the RBS would be upstream of any coding sequence inserted into the MCS. Another alternative is to include a ribosome binding site within the MCS, in which case the choice of restriction enzymes used to cut within the MCS will determine whether the RBS is retained, and in what relation to, the inserted sequences. A further alternative is to include a RBS within the polynucleotide sequence that is to be inserted into the expression construct at the MCS, preferably in the proper relation to any coding sequences to stimulate initiation of translation from the transcribed messenger RNA.

Expression from constitutive promoters. Expression constructs of the disclosure can also comprise coding sequences that are expressed from constitutive promoters. Unlike inducible promoters, constitutive promoters initiate continual gene product production under most growth conditions. One example of a constitutive promoter is that of the Tn3 bla gene, which encodes beta-lactamase and is responsible for the ampicillin-resistance (AmpR) phenotype conferred on the host cell by many plasmids, including pBR322 (ATCC 31344), pACYCIW (ATCC 37031), and pBAD24 (ATCC 87399). Another constitutive promoter that can be used in expression constructs is the promoter for the E. coli lipoprotein gene, Ipp, which is located at positions 1755731-1755406 (plus strand) in E. coli K-12 substrain MG1655 (Inouye and Inouye, Nucleic Acids Res 1985 May 10; 13 (9): 3101-3110). A further example of a constitutive promoter that has been used for heterologous gene expression in E. coli is the trpLEDCBA promoter, located at positions 1321169-1321133 (minus strand) in E. coli K-12 substrain MG1655 (Windass et al., Nucleic Acids Res 1982 Nov. 11; 10 (21): 6639-6657). Constitutive promoters can be used in expression constructs for the expression of selectable markers, as described herein, and also for the constitutive expression of other gene products useful for the coexpression of the desired product. For example, transcriptional regulators of the inducible promoters, such as AraC, PrpR, RhaR, and XylR, if not expressed from a bidirectional inducible promoter, can alternatively be expressed from a constitutive promoter, on either the same expression construct as the inducible promoter they regulate, or a different expression construct. Similarly, gene products useful for the production or transport of the inducer, such as PrpEC, AraE, or Rha, or proteins that modify the reduction-oxidation environment of the cell, as a few examples, can be expressed from a constitutive promoter within an expression construct. Gene products useful for the production of coexpressed gene products, and the resulting desired product, also include chaperone proteins, cofactor transporters, etc.

Signal Peptides. Polypeptide gene products expressed or coexpressed by the methods of the disclosure can contain signal peptides or lack them, depending on whether it is desirable for such gene products to be exported from the host cell cytoplasm into the periplasm, or to be retained in the cytoplasm, respectively. Signal peptides (also termed signal sequences, leader sequences, or leader peptides) are characterized structurally by a stretch of hydrophobic amino acids, approximately five to twenty amino acids long and often around ten to fifteen amino acids in length, that has a tendency to form a single alpha-helix. This hydrophobic stretch is often immediately preceded by a shorter stretch enriched in positively charged amino acids (particularly lysine). Signal peptides that are to be cleaved from the mature polypeptide typically end in a stretch of amino acids that is recognized and cleaved by signal peptidase. Signal peptides can be characterized functionally by the ability to direct transport of a polypeptide, either co-translationally or post-translationally, through the plasma membrane of prokaryotes (or the inner membrane of gram negative bacteria like E. coli), or into the endoplasmic reticulum of eukaryotic cells. The degree to which a signal peptide enables a polypeptide to be transported into the periplasmic space of a host cell like E. coli, for example, can be determined by separating periplasmic proteins from proteins retained in the cytoplasm, using a method such as described in Example 12 of WO/2016/205570.

The following is a description of inducible promoters that can be used in expression constructs for expression or coexpression of gene products, along with some of the genetic modifications that can be made to host cells that contain such expression constructs. Examples of these inducible promoters and related genes are, unless otherwise specified, from Escherichia coli (E. coli) strain MG1655 (American Type Culture Collection deposit ATCC 700926), which is a substrain of E. coli K-12 (American Type Culture Collection deposit ATCC 10798). Table 1 of WO/2016/205570 lists the genomic locations, in E. coli MG1655, of the nucleotide sequences for these examples of inducible promoters and related genes. Nucleotide and other genetic sequences, referenced by genomic location as in Table 1 of WO/2016/205570, are expressly incorporated by reference herein. Additional information about E. coli promoters, genes, and strains described herein can be found in many public sources, including the online EcoliWiki resource, located at ecoliwiki.net.

Arabinose promoter. (As used herein, ‘arabinose’ means L-arabinose.) Several E. coli operons involved in arabinose utilization are inducible by arabinose—araBAD, araC, arciE, and araFGH—but the terms ‘arabinose promoter’ and ‘ara promoter’ are typically used to designate the araBAD promoter. Several additional terms have been used to indicate the E. coli araBAD promoter, such as Para, ParaB, ParaBAD, and PBAD—The use herein of ‘ara promoter’ or any of the alternative terms given above, means the E. coli araBAD promoter. As can be seen from the use of another term, ‘araC-araBAD promoter’, the araBAD promoter is considered to be part of a bidirectional promoter, with the araBAD promoter controlling expression of the araBAD operon in one direction, and the araC promoter, in close proximity to and on the opposite strand from the araBAD promoter, controlling expression of the araC coding sequence in the other direction. The AraC protein is both a positive and a negative transcriptional regulator of the araBAD promoter. In the absence of arabinose, the AraC protein represses transcription from PBAD, but in the presence of arabinose, the AraC protein, which alters its conformation upon binding arabinose, becomes a positive regulatory element that allows transcription from PBAD—The araBAD operon encodes proteins that metabolize L-arabinose by converting it, through the intermediates L-ribulose and L-ribulose-phosphate, to D-xylulose-5-phosphate. For the purpose of maximizing induction of expression from an arabinose-inducible promoter, it is useful to eliminate or reduce the function of AraA, which catalyzes the conversion of L-arabinose to L-ribulose, and optionally to eliminate or reduce the function of at least one of AraB and AraD, as well. Eliminating or reducing the ability of host cells to decrease the effective concentration of arabinose in the cell, by eliminating or reducing the cell's ability to convert arabinose to other sugars, allows more arabinose to be available for induction of the arabinose-inducible promoter. The genes encoding the transporters which move arabinose into the host cell are araE, which encodes the low-affinity L-arabinose proton symporter, and the araFGH operon, which encodes the subunits of an ABC superfamily high-affinity L-arabinose transporter. Other proteins which can transport L-arabinose into the cell are certain mutants of the LacY lactose permease: the LacY (AlWC) and the LacY (AlWV) proteins, having a cysteine or a valine amino acid instead of alanine at position 177, respectively (Morgan-Kiss et al., Proc Natl Acad Sci USA 2002 May 28; 99 (11): 7373-7377). In order to achieve homogenous induction of an arabinose-inducible promoter, it is useful to make transport of arabinose into the cell independent of regulation by arabinose. This can be accomplished by eliminating or reducing the activity of the AraFGH transporter proteins and altering the expression of araE so that it is only transcribed from a constitutive promoter. Constitutive expression of araE can be accomplished by eliminating or reducing the function of the native araE gene, and introducing into the cell an expression construct which includes a coding sequence for the AraE protein expressed from a constitutive promoter. Alternatively, in a cell lacking AraFGH function, the promoter controlling expression of the host cell's chromosomal araE gene can be changed from an arabinose-inducible promoter to a constitutive promoter. In similar manner, as additional alternatives for homogenous induction of an arabinose-inducible promoter, a host cell that lacks AraE function can have any functional AraFGH coding sequence present in the cell expressed from a constitutive promoter. As another alternative, it is possible to express both the araE gene and the araFGH operon from constitutive promoters, by replacing the native araE and araFGH promoters with constitutive promoters in the host chromosome. It is also possible to eliminate or reduce the activity of both the AraE and the AraFGH arabinose transporters, and in that situation to use a mutation in the LacY lactose permease that allows this protein to transport arabinose. Since expression of the lacY gene is not normally regulated by arabinose, use of a LacY mutant such as LacY (A177C) or LacY (A177V), will not lead to the ‘all or none’ induction phenomenon when the arabinose-inducible promoter is induced by the presence of arabinose. Because the LacY (A177C) protein appears to be more effective in transporting arabinose into the cell, use of polynucleotides encoding the LacY (A177C) protein is preferred to the use of polynucleotides encoding the LacY (A177V) protein.

Propionate promoter. The ‘propionate promoter’ or ‘prp promoter’ is the promoter for the E. coli prpBCDE operon, and is also called PPΦB. Like the ara promoter, the prp promoter is part of a bidirectional promoter, controlling expression of the prpBCDE operon in one direction, and with the prpR promoter controlling expression of the prpR coding sequence in the other direction. The PrpR protein is the transcriptional regulator of the prp promoter, and activates transcription from the prp promoter when the PrpR protein binds 2-methylcitrate (‘2-MC’). Propionate (also called propanoate) is the ion, CH3CH2COO—, of propionic acid (or ‘propanoic acid’), and is the smallest of the ‘fatty’ acids having the general formula H(CH2), COOH that shares certain properties of this class of molecules: producing an oily layer when salted out of water and having a soapy potassium salt. Commercially available propionate is generally sold as a monovalent cation salt of propionic acid, such as sodium propionate (CH3CH2COONa), or as a divalent cation salt, such as calcium propionate (Ca(CH3CH2COO)2). Propionate is membrane-permeable and is metabolized to 2-MC by conversion of propionate to propionyl-CoA by PrpE (propionyl-CoA synthetase), and then conversion of propionyl-CoA to 2-MC by PrpC (2-methylcitrate synthase). The other proteins encoded by the prpBCDE operon, PrpD (2-methylcitrate dehydratase) and PrpB (2-methylisocitrate lyase), are involved in further catabolismof 2-MC into smaller products such as pyruvate and succinate. In order to maximize induction of a propionate-inducible promoter by propionate added to the cell growth medium, it is therefore desirable to have a host cell with PrpC and PrpE activity, to convert propionate into 2-MC, but also having eliminated or reduced PrpD activity, and optionally eliminated or reduced PrpB activity as well, to prevent 2-MC from being metabolized. Another operon encoding proteins involved in 2-MC biosynthesis is the scpA-argK-scpBC operon, also called the sbm-yg/DGH operon. These genes encode proteins required for the conversion of succinate to propionyl-CoA, which can then be converted to 2-MC by PrpC. Elimination or reduction of the function of these proteins would remove a parallel pathway for the production of the 2-MC inducer, and thus might reduce background levels of expression of a propionate-inducible promoter, and increase sensitivity of the propionate-inducible promoter to exogenously supplied propionate. It has been found that a deletion of sbm-ygfD-ygfG-ygfH-ygfl, introduced into E. coli BL21(DE3) to create strain JSB (Lee and Keasling, “A propionate-inducible expression system for enteric bacteria”, Appl Environ Microbiol 2005 November; 71 (11): 6856-6862), was helpful in reducing background expression in the absence of exogenously supplied inducer, but this deletion also reduced overall expression from the prp promoter in strain JSB. It should be noted, however, that the deletion sbm-ygfD-ygfG-ygfH-ygfl also apparently affects ygfl, which encodes a putative LysR-family transcriptional regulator of unknown function. The genes sbm-yg/DGH are transcribed as one operon, and ygfl is transcribed from the opposite strand. The 3′ ends of the ygfti and ygfl coding sequences overlap by a few base pairs, so a deletion that takes out all of the sbm-yg/DGH operon apparently takes out ygfl coding function as well. Eliminating or reducing the function of a subset of the sbm-ygfDGH gene products, such as YgfG (also called ScpB, methylmalonyl-CoA decarboxylase), or deleting the majority of the sbm-yg/DGH (or scpA-argK-scpBC) operon while leaving enough of the 3′ end of the ygfli (or scpC) gene so that the expression of ygfl is not affected, could be sufficient to reduce background expression from a propionate-inducible promoter without reducing the maximal level of induced expression.

Rhamnose promoter. (As used herein, ‘rhamnose’ means L-rhamnose.) The ‘rhamnose promoter’ or ‘rha promoter’, or PrhaSR, is the promoter for the E. coli rhaSR operon. Like the ara and prp promoters, the rha promoter is part of a bidirectional promoter, controlling expression of the rhaSR operon in one direction, and with the rhaBAD promoter controlling expression of the rhaBAD operon in the other direction. The rha promoter, however, has two transcriptional regulators involved in modulating expression: RhaR and RhaS. The RhaR protein activates expression of the rhaSR operon in the presence of rhamnose, while RhaS protein activates expression of the L-rhamnose catabolic and transport operons, rhaBAD and rhaT, respectively (Wickstrum et al, J Bacteriol 2010 January; 192 (1): 225-232). Although the RhaS protein can also activate expression of the rhaSR operon, in effect RhaS negatively autoregulates this expression by interfering with the ability of the cyclic AMP receptor protein (CRP) to coactivate expression with RhaR to a much greater level. The rhaBAD operon encodes the rhamnose catabolic proteins RhaA (L-rhamnose isomerase), which converts L-rhamnose to L-rhamnulose; RhaB (rhamnulokinase), which phosphorylates L-rhamnulose to form L-rhamnulose-1-P; and RhaD (rhamnulose-1-phosphate aldolase), which converts L-rhamnulose-1-P to L-lactaldehyde and DHAP (dihydroxy acetone phosphate). To maximize the amount of rhamnose in the cell available for induction of expression from a rhamnose-inducible promoter, it is desirable to reduce the amount of rhamnose that is broken down by catalysis, by eliminating or reducing the function of RhaA, or optionally of RhaA and at least one of RhaB and RhaD. E. coli cells can also synthesize L-rhamnose from alpha-D-glucose-1-P through the activities of the proteins RmlA, RmlB, RmlC, and RmlD (also called RfbA, RfbB, RfbC, and RfbD, respectively) encoded by the rmlBDACX (or rfbBDACX) operon. To reduce background expression from a rhamnose-inducible promoter, and to enhance the sensitivity of induction of the rhamnose-inducible promoter by exogenously supplied rhamnose, it could be useful to eliminate or reduce the function of one or more of the RmlA, RmlB, RmlC, and

RmlD proteins. L-rhamnose is transported into the cell by RhaT, the rhamnose permease or L-rhamnose: proton symporter. As noted above, the expression of RhaT is activated by the transcriptional regulator RhaS. To make expression of RhaT independent of induction by rhamnose (which induces expression of RhaS), the host cell can be altered so that all functional RhaT coding sequences in the cell are expressed from constitutive promoters. Additionally, the coding sequences for RhaS can be deleted or inactivated, so that no functional RhaS is produced. By eliminating or reducing the function of RhaS in the cell, the level of expression from the rhaSR promoter is increased due to the absence of negative autoregulation by RhaS, and the level of expression of the rhamnose catalytic operon rhaBAD is decreased, further increasing the ability of rhamnose to induce expression from the rha promoter.

Xylose promoter. (As used herein, ‘xylose’ means D-xylose.) The xylose promoter, or ‘xyl promoter’, or PxyiA, means the promoter for the E. coli xylAB operon. The xylose promoter region is similar in organization to other inducible promoters in that the xylAB operon and the xylFGHR operon are both expressed from adjacent xylose-inducible promoters in opposite directions on the E. coli chromosome (Song and Park, J Bacteriol. 1997 November; 179 (22): 7025-7032). The transcriptional regulator of both the PxyiA and PxyiF promoters is XyIR, which activates expression of these promoters in the presence of xylose. The xylR gene is expressed either as part of the xylFGHR operon or from its own weak promoter, which is not inducible by xylose, located between the xylH and xylR protein-coding sequences. D-xylose is catabolized by XylA (D-xylose isomerase), which converts D-xylose to D-xylulose, which is then phosphorylated by XylB (xylulokinase) to form D-xylulose-5-P. To maximize the amount of xylose in the cell available for induction of expression from a xylose-inducible promoter, it is desirable to reduce the amount of xylose that is broken down by catalysis, by eliminating or reducing the function of at least XylA, or optionally of both XylA and XylB. The xylFGHR operon encodes XylF, XylG, and XylH, the subunits of an ABC super-family high-affinity D-xylose transporter. The xylE gene, which encodes the E. coli low-affinity xylose-proton symporter, represents a separate operon, the expression of which is also inducible by xylose. To make expression of a xylose transporter independent of induction by xylose, the host cell can be altered so that all functional xylose transporters are expressed from constitutive promoters. For example, the xylFGHR operon could be altered so that the xylFGH coding sequences are deleted, leaving XylR as the only active protein expressed from the xylose-inducible PxyiF promoter, and with the xylE coding sequence expressed from a constitutive promoter rather than its native promoter. As another example, the xylR coding sequence is expressed from the PxyiA or the promoter in an expression construct, while either the xylFGHR operon is deleted and xylE is constitutively expressed, or alternatively an xylFGH operon (lacking the xylR coding sequence since that is present in an expression construct) is expressed from a constitutive promoter and the xylE coding sequence is deleted or altered so that it does not produce an active protein.

Lactose promoter. The term ‘lactose promoter’ refers to the lactose-inducible promoter for the lacZYA operon, a promoter which is also called lacZpl; this lactose promoter is located at ca. 365603-365568 (minus strand, with the NA polymerase binding (‘-35’) site at ca. 365603-365598, the Pribnow box (‘-10’) at 365579-365573, and a transcription initiation site at 365567) in the genomic sequence of the E. coli K-12 substrain MG1655 (NCBI Reference Sequence NC 000913.2, 11 Jan. 2012). In some embodiments, inducible coexpression systems of the disclosure can comprise a lactose-inducible promoter such as the lacZYA promoter. In other embodiments, the inducible coexpression systems of the disclosure comprise one or more inducible promoters that are not lactose-inducible promoters.

Alkaline phosphatase promoter. The terms ‘alkaline phosphatase promoter’ and ‘phoA promoter’ refer to the promoter for the phoApsiF operon, a promoter which is induced under conditions of phosphate starvation. The phoA promoter region is located at ca. 401647-401746 (plus strand, with the Pribnow box (‘−10’) at 401695-401701 (Kikuchi et al., Nucleic Acids Res 1981 Nov. 11; 9 (21): 5671-5678)) in the genomic sequence of the E. coli K-12 substrain MG1655 (NCBI Reference Sequence NC 000913.3, 16 Dec. 2014). The transcriptional activator for the phoA promoter is PhoB, a transcriptional regulator that, along with the sensor protein PhoR, forms a two-component signal transduction system in E. coli. PhoB and PhoR are transcribed from the phoBR operon, located at ca. 417050-419300 (plus strand, with the PhoB coding sequence at 417,142-417,831 and the PhoR coding sequence at 417,889-419,184) in the genomic sequence of the E. coli K-12 substrain MG1655 (NCBI Reference Sequence NC 000913.3, 16 Dec. 2014). The phoA promoter differs from the inducible promoters described above in that it is induced by the lack of a substance-intracellular phosphate-rather than by the addition of an inducer. For this reason the phoA promoter is generally used to direct transcription of gene products that are to be produced at a stage when the host cells are depleted for phosphate, such as the later stages of fermentation. In some embodiments, inducible coexpression systems of the disclosure can comprise a phoA promoter. In other embodiments, the inducible coexpression systems of the disclosure comprise one or more inducible promoters that are not phoA promoters.

As described herein, it may be advantageous or desirable to remove (e.g., by way of an inducible or constitutive “curing” mechanism) an expression construct described herein, e.g., if the cell line harboring the expression construct is or will be used for commercial purposes. Thus, in some embodiments, the expression construct may comprise a “kill switch.” For example, in embodiment, the expression construct includes a temperature-sensitive origin of replication. Additional curing methods are known in the art and include using detergents and intercalating agents, drugs and antibiotics (Buckner, M. M. C., et al., FEMS Microbiology Reviews, fuy031,42, 2018, 781-804).

Before the present disclosure is further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

It must be noted that as used herein and in the appended claims, the singular forms “a,” “and,” and “the” include plural referents unless the context clearly dictates otherwise. Thus, for example, reference to “a conformation switching probe” includes a plurality of such conformation switching probes and reference to “the microfluidic device” includes reference to one or more microfluidic devices and equivalents thereof known to those skilled in the art, and so forth. It is further noted that the claims may be drafted to exclude any element, e.g., any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order which is logically possible. This is intended to provide support for all such combinations.

### EXAMPLES

### Example 1

### Growth/No Growth Selection Assay for Higher Expression E. coli Strains

The present Example describes and performs a simple growth/no growth selection strategy to identify higher soluble protein expression E. coli strains in a mixed population (library).

An E. coli strain harboring a plasmid encoding an arabinose promoter driven expression of mCherry-FolA fusion protein was used herein to show (1) Trimethoprim, by itself, has very limited selectable dynamic range and (2) the addition of sulfamethoxazole sensitizes the mCherry-FolA strain to trimethoprim.

Strains: Strain #1. E. coli harboring plasmid encoding antibiotic target gene (folA) fusion under the control of pBAD promoter. Strain #2. Control E. coli harboring plasmid encoding a different antibiotic target gene (murA).

Experiment: Prepare trimethoprim antibiotic gradient plates containing arabinose. The antibiotic gradient will be identical across the plates. However, the concentration of the second antibiotic agent (sulfamethoxazole) will be uniform in each plate but different across the plates. Inoculate strains across the antibiotic gradient. The length of growth is a measure of relative antibiotic resistance. For the strain harboring the pBAD plasmid with the folA gene, the length of growth would be inversely correlated to the sulfamethoxazole concentration: The plate containing the highest sulfamethoxazole concentration should have the shortest length of growth.

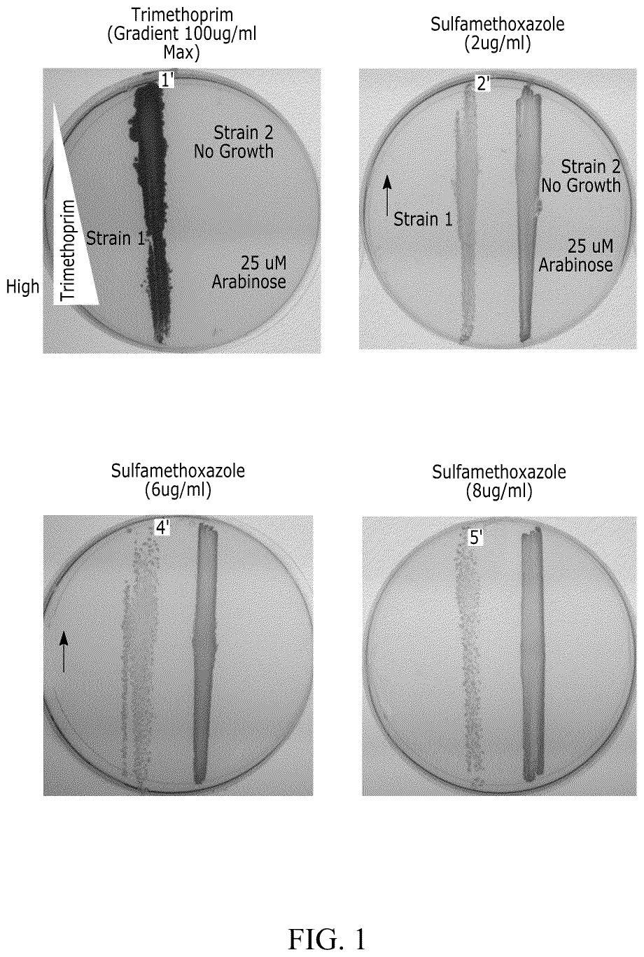

As shown in FIG. 1, cells harboring the mCherry-FolA fusion are highly resistant to trimethoprim. FIG. 1 shows that resistance to 100 ug/ml trimethoprim (mCherry-FolA) is dependent on arabinose and mCherry-FolA fusion. Sulfamethoxazole at up to 16 ug/ml did not inhibit growth.

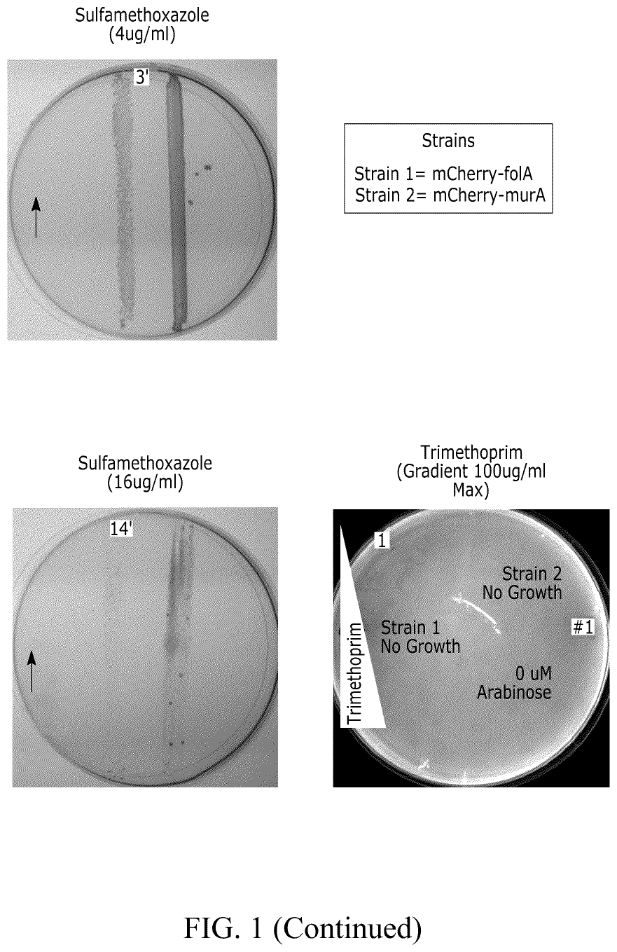

As shown in FIG. 2, synergy with sulfamethoxazole improves trimethoprim dynamic range. FIG. 2 shows that trimethoprim has strong synergy with sulfamethoxazole: sensitivity to trimethoprim is restored with >2 ug/ml sulfamethoxazole.

### Example 2

### Soluble Protein Selection Assay-Insulin-FolA Fusion

The present Example describes and performs the assay from Example 1 using two insulin-FolA fusion constructs (insulin variants) and using two E. coli chaperone libraries.

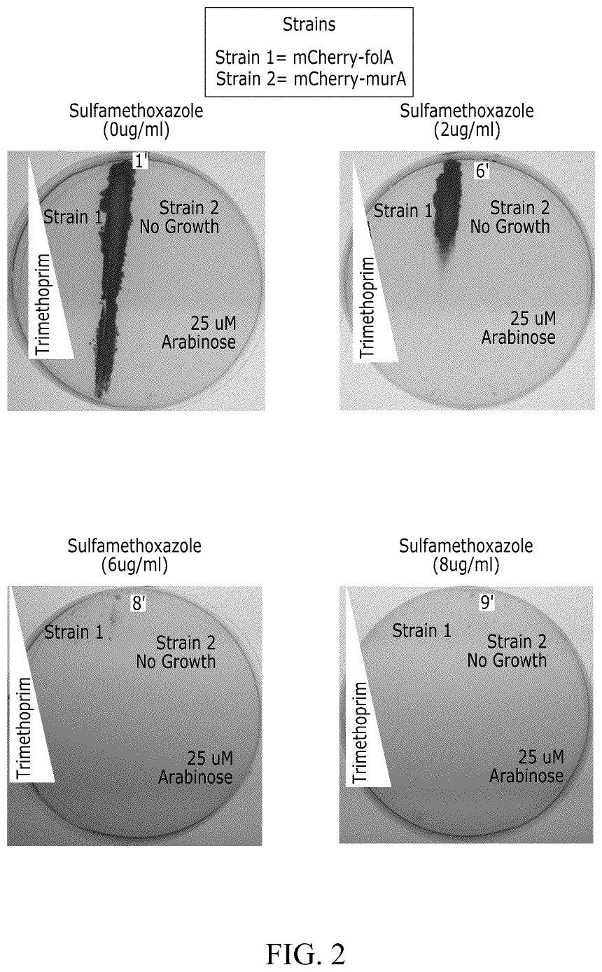

Agar plates were prepared that covered a matrix of the two agents, trimethoprim and sulfamethoxazole, in combination as described herein and in Example 1 and FIGS. 1-2. The combination matrix is necessary because the actual concentration of antibiotic tolerated by the best performing strain in the library is not known. Two control strains bearing an empty plasmid (without chaperone) and the experimental strains with the chaperone plasmids were each plated individually on the entire set of plates covered by the combination matrix (e.g., matrix of trimethoprim-sulfamethoxazole), as shown in FIG. 3. Approximately 300 million cells per plate from each of the six strains were plated individually on all 16 plates. The plates were incubated for 5 days before final analysis.

After five days incubation, differences were observed between the control and the experimental strains in some of the agar plates. These differences are either subtle, where the experimental plates show larger colonies than the control, or striking, where growth was observed only in the experimental plates and not in the control. For each of the two insulin-FolA fusion/chaperone plasmid pair, seven positive hits were randomly picked for further characterization using the soluble insulin HiPrBind assay (See, e.g., WO/2021/163349). Of the seven strains picked for each of two insulin-FolA fusion, two strains showed increased amounts of soluble insulin compared to the control strain.

In conclusion, the FolA selection assay enables a growth/no growth selection for strains that produced higher quantities of soluble protein of interest-FolA fusion. Additionally, a gradation of expression strains, from low to very high, can be obtained by modifying the selection conditions where the concentrations of the synergistic antibiotics are increased. Lastly, large libraries containing multiple billions of cells can be tested in parallel to identify the best performing subset of strains.

### Example 3

### Soluble Protein Selection Assay-Alpha Fetoprotein

The present Example describes and performs the assays described above that incorporates a solid phase assay to further analyze the population of cells. The solid phase assay is described herein and, for example, in PCT/US22/53107.

Applying the assay as described in the above Examples on two different target proteins (insulin and alpha fetoprotein) showed that the assay yielded several hundred growing colonies (candidates) after selection on selective plates. The ideal goal would be to test all candidates to determine whether it is a true outperformer or a mutant that picked up an escape mechanism. Here, a solid phase assay was employed to test the entire growing population to identify outperformers that actually produced more of the fusion protein than the background control.

### Determination of Relative Expression for Soluble Alpha Fetoprotein by Capillary Immunoblot Using the Protein Simple Jess Automated Instrument

Wet cell paste from 100 μL of culture was resuspended in lysis buffer (50 mM Tris-HCl, 200 mM Sodium Chloride, 10.0 g/L Octyl Glucoside, 2400 U/mL Lysozyme, 2 U/mL Benzonase, pH 7.2) and lysed on ice for one hour. Following lysis, the samples were diluted in 0.1×Sample Buffer (Protein Simple) to an OD600 of 0.008 and centrifuged at 3300×g for 30 minutes at 4° C. Following centrifugation, the samples were mixed with fluorescent master mix (Protein Simple) containing 100 mM iodoacetamide to an OD600 of 0.0064, vortexed, and heated to 95° C. for 5 minutes. Prepared samples were loaded into a 12-230 kDa Separation Module plate (Protein Simple), in addition to the rabbit monoclonal anti-alpha 1 Fetoprotein primary antibody (abcam item #ab 169552) and reagents required to perform a chemiluminescent immunoassay. The Separation Module plate was loaded into the Jess along with a 25-capillary cartridge and run using the default chemiluminescent detection method. Simple Western assay results were analyzed using the Protein Simple Compass for Simple Western software.

### Case Study: Alpha Fetoprotein

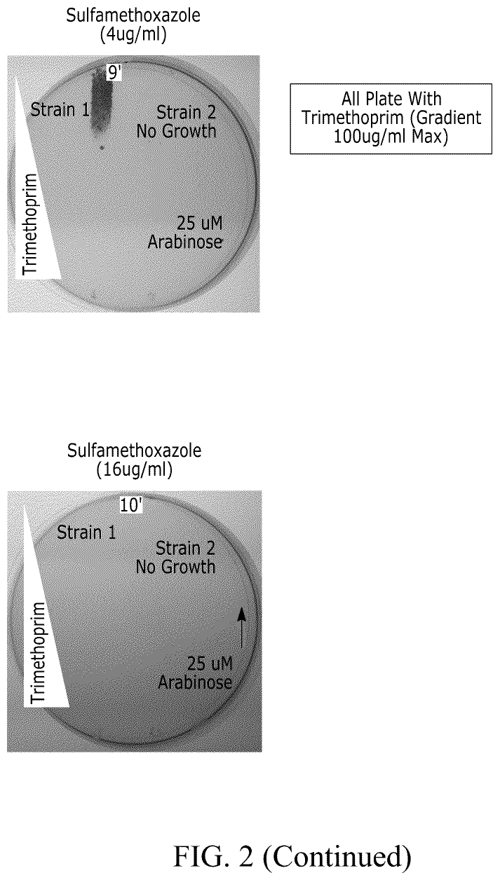

A plasmid library was constructed with the following characteristics. (FIG. 4) The arabinose promoter controls the target protein (alpha fetoprotein) which was genetically fused in frame at the C-terminus to folA. In addition, plasmid was designed to harbor two chaperones, driven by the propionate promoter, that are randomly chosen from a pool of ˜1200 unique chaperone genes. The theoretical diversity is 1.44 million unique combinations (1200×1200).

A set of agar plates were prepared containing 10 μg/ml kanamycin, 125 mM arabinose, 1 mM propionate and one of 64 different combinations of trimethoprim/sulfamethoxazole according to Table B below.

TABLE B

| | | Trimethoprim (μg/ml) | | | | |
|---|---|---|---|---|---|---|
| | | 1 | 2 | 4 | 8 | 16 |
| sulfamethoazole | 0 | TRM1_Sulfa0 | TRM2_Sulfa0 | TRM4_Sulfa0 | TRM8_Sulfa0 | TRM16_Sulfa0 |
| (μg/ml) | 1 | TRM1_Sulfa1 | TRM2_Sulfa1 | TRM4_Sulfa1 | TRM8_Sulfa1 | TRM16_Sulfa1 |
| | 2 | TRM1_Sulfa2 | TRM2_Sulfa2 | TRM4_Sulfa2 | TRM8_Sulfa2 | TRM16_Sulfa2 |
| | 4 | TRM1_Sulfa4 | TRM2_Sulfa4 | TRM4_Sulfa4 | TRM8_Sulfa4 | TRM16_Sulfa4 |
| | 8 | TRM1_Sulfa8 | TRM2_Sulfa8 | TRM4_Sulfa8 | TRM8_Sulfa8 | TRM16_Sulfa8 |
| | 16 | TRM1_Sulfa16 | TRM2_Sulfa16 | TRM4_Sulfa16 | TRM8_Sulfa16 | TRM16_Sulfa16 |
| | 32 | TRM1_Sulfa32 | TRM2_Sulfa32 | TRM4_Sulfa32 | TRM8_Sulfa32 | TRM16_Sulfa32 |
| | 64 | TRM1_Sulfa64 | TRM2_Sulfa64 | TRM4_Sulfa64 | TRM8_Sulfa64 | TRM16_Sulfa64 |

| | | Trimethoprim (μg/ml) | | |
|---|---|---|---|---|
| | | 32 | 64 | 128 |
| sulfamethoazole | 0 | TRM32_Sulfa0 | TRM64_Sulfa0 | TRM128_Sulfa0 |
| (μg/ml) | 1 | TRM32_Sulfa1 | TRM64_Sulfa1 | TRM128_Sulfa1 |
| | 2 | TRM32_Sulfa2 | TRM64_Sulfa2 | TRM128_Sulfa2 |
| | 4 | TRM32_Sulfa4 | TRM64_Sulfa4 | TRM128_Sulfa4 |
| | 8 | TRM32_Sulfa8 | TRM64_Sulfa8 | TRM128_Sulfa8 |
| | 16 | TRM32_Sulfa16 | TRM64_Sulfa16 | TRM128_Sulfa16 |
| | 32 | TRM32_Sulfa32 | TRM64_Sulfa32 | TRM128_Sulfa32 |
| | 64 | TRM32_Sulfa64 | TRM64_Sulfa64 | TRM128_Sulfa64 |

Approximately 300 million cells of this library were plated on each of 64 plates. A control strain, harboring the exact plasmid as the library except the plasmid contained no chaperone genes, was also plated on each of 64 plates. The entire collection of plates was incubated at 30° C. for 1 week. When the plates were examined, only a subset of plates (in the bold sectors in Table C) contain visible colonies. The others either had no change in growth compared to day 0 or a lawn of bacteria with no robust colony present.

TABLE C

| | | Trimethoprim (μg/ml) | | | | |
|---|---|---|---|---|---|---|
| | | 1 | 2 | 4 | 8 | 16 |
| sulfamethoazole | 0 | TRM1_Sulfa0 | TRM2_Sulfa0 | TRM4_Sulfa0 | TRM8_Sulfa0 | TRM16_Sulfa0 |
| (μg/ml) | 1 | TRM1_Sulfa1 | TRM2_Sulfa1 | TRM4_Sulfa1 | TRM8_Sulfa1 | TRM16_Sulfa1 |
| | 2 | TRM1_Sulfa2 | TRM2_Sulfa2 | TRM4_Sulfa2 | TRM8_Sulfa2 | TRM16_Sulfa2 |
| | 4 | TRM1_Sulfa4 | TRM2_Sulfa4 | TRM4_Sulfa4 | TRM8_Sulfa4 | TRM16_Sulfa4 |
| | 8 | TRM1_Sulfa8 | TRM2_Sulfa8 | TRM4_Sulfa8 | TRM8_Sulfa8 | TRM16_Sulfa8 |
| | 16 | TRM1_Sulfa16 | TRM2_Sulfa16 | TRM4_Sulfa16 | TRME_Sulfa16 | TRM16_Sulfa16 |
| | 32 | TRM1_Sulfa32 | TRM2_Sulfa32 | TRM4_Sulfa32 | TRM8_Sulfa32 | TRM16_Sulfa32 |
| | 64 | TRM1_Sulfa64 | TRM2_Sulfa64 | TRM4_Sulfa64 | TRM8_Sulfa64 | TRM16_Sulfa64 |

| | | Trimethoprim (μg/ml) | | |
|---|---|---|---|---|
| | | 32 | 64 | 128 |
| sulfamethoazole | 0 | TRM32_Sulfa0 | TRM64_Sulfa0 | TRM128_Sulfa0 |
| (μg/ml) | 1 | TRM32_Sulfa1 | TRM64_Sulfa1 | TRM128_Sulfa1 |
| | 2 | TRM32_Sulfa2 | TRM64_Sulfa2 | TRM128_Sulfa2 |
| | 4 | TRM32_Sulla4 | TRM64_Sulfa4 | TRM128_Sulfa4 |
| | 8 | TRM32_Sulfa8 | TRM64_Sulfa8 | TRM128_Sulfa8 |
| | 16 | TRM32_Sulfa16 | TRM64_Sulfa16 | TRM128_Sulfa16 |
| | 32 | TRM32_Sulfa32 | TRM64_Sulfa32 | TRM128_Sulfa32 |
| | 64 | TRM32_Sulfa64 | TRM64_Sulfa64 | TRM128_Sulfa64 |

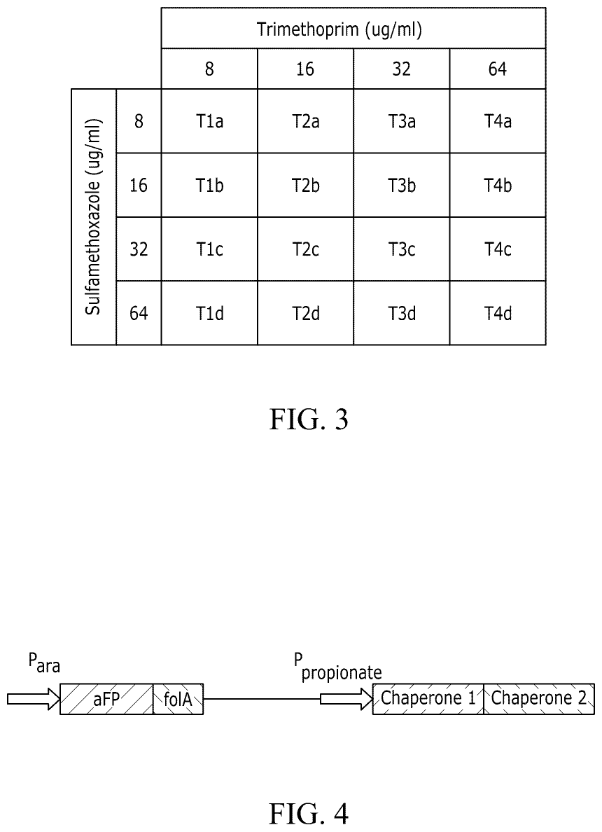

Example of plates with colonies after 1 week incubation are shown in FIG. 5.

Eight of the 9 plates were analyzed by solid phase assay.

### Round 1 Solid Phase Assay

Nitrocellulose membrane (Cytiva Protran BA 83 Cat. No. 10401316) is layered on the surface of the agar. A sewing needle was used to apply latex paint to the agar to be used for positional alignment of the agar plate with the image derived from the nitrocellulose membrane after processing. This was done by dipping the needled into a brightly colored latex paint then using the paint-covered needle tip to puncture the membrane, through the agar, leaving some of the paint in the agar and a puncture mark on the membrane. The membrane was then gently lifted from the agar surface and placed on a Whatman paper (Cytiva cat. No. 1001-090) with the side containing the bound cells facing up. In all subsequent steps, the membrane is always placed with the cell side facing up. The Whatman paper was used to remove the excess paint from the nitrocellulose membrane. The nitrocellulose was then transferred to a clean dish. A 2 ml solution of the fixative solution [2.6% (w/v) paraformaldehyde, 0.04% (w/v) glutaraldehyde, 32.25 mM Na₃PO₄ pH 7.4] was applied to the membrane and allowed to incubate at room temperature for 3 minutes. Afterwards, the membrane was washed 3 times with 1×PBS [135 mM NaCl, 2.7 mM KCl, 11 mM Phosphate Buffer pH 7.4]. The membrane was then incubated overnight in blocking solution [0.1M NaHCO3 pH 8.6, 1 mM EDTA, 5 mg/ml bovine serum albumin (fraction V), 1 μM biotin]. The next day, the membrane was transferred to a fresh dish and treated with 5 ml lysozyme solution [Millipore Sigma cat no 71110-4, diluted 10,000 fold in 1× Immunoassay buffer (PerkinElmer cat no. AL000F)] for 10 minutes. Subsequently, the membrane was transferred to a fresh dish and incubated with detection reagent for alpha fetoprotein [0.25 nM anti-alpha fetoprotein (abcam cat no. ab130748), 0.25 nM anti-rabbit antibody-alkaline phosphatase (Millipore Sigma cat no. A2556)] overnight. The next day, the membrane was washed 6-8 times, each time by draining excess fluid and transferring the membrane to a fresh dish containing 20-50 ml of 1× wash buffer (Azure Biosystems cat. no. AC2113). After the final wash, the membrane was treated with 2 ml of alkaline phosphatase substrate (SeraCare KPL PhosphaGLO, cat. No. 5430-0055) for ˜3 min. The membrane was drained and placed on Whatman paper (Cytiva cat. No. 1001-090). The puncture marks on the membrane are highlighted by hand using a black pen. The membrane was then placed inside the imaging chamber of the Azure 600 imager (Azure Biosystems) and imaged for (1) fluorescence (Excitation 732/Emission 832) and (2) chemiluminescence. The fluorescence image captured the pen markings and was merged with the chemiluminescent image using the Azure Biosystems image capture software. The fluorescent image contains the markings to align the chemiluminescence image with the agar plate. The merged image was printed onto a clear transparency film using a laser printer.

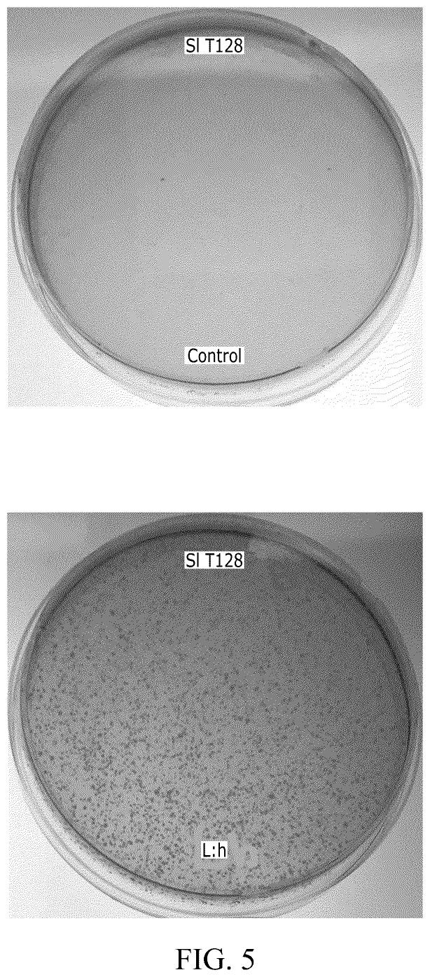

Example of images obtained are shown in FIG. 6.

A review of the 8 membranes analyzed (bold lettering in the matrix below in Table D) showed that only three of the 8 membranes contained positive hits. The three membranes that contained the positive hits were derived from agar plates that contained trimethoprim/sulfamethoxazole combinations from the blue colored box in the matrix below. This result suggests that only the colonies that appear at the highest tolerated concentration of trimethoprim/sulfamethoxazole contain true outperformers for this selection strategy.

### Recovery and Characterization of Hits

50 hits were picked from the corresponding plate using the images obtained to guide hit recovery. The merged image (fluorescent+chemiluminescent images) contains both the location of the hits and the alignment information to recovery live hits from the source agar plate. The agar is aligned with printed transparency film using the paint marks in the agar and the dots captured by fluorescence imaging on the transparency film. Putative hits are recovered using the blunt end of a sterile loop, by gently touching the surface of the agar with the loop and then subsequently touching the surface of a fresh induction plate [LB/Kan (10 μg/ml)/arabinose (125 μM)/propionate (1 mM)] grided into 8 sectors. Each putative hit is given one sector. In each sector, a sterile loop is then used to streak out the initial inoculum to separate for single colonies by spread out the recovered E. coli. The induction plates are incubated at 30° C. for 2 days. The plates are then processed using the exact procedure described for round 1 solid phase hit identification.

### Round 2 Results

### Example Image of Test Plate Compared to Control Plate

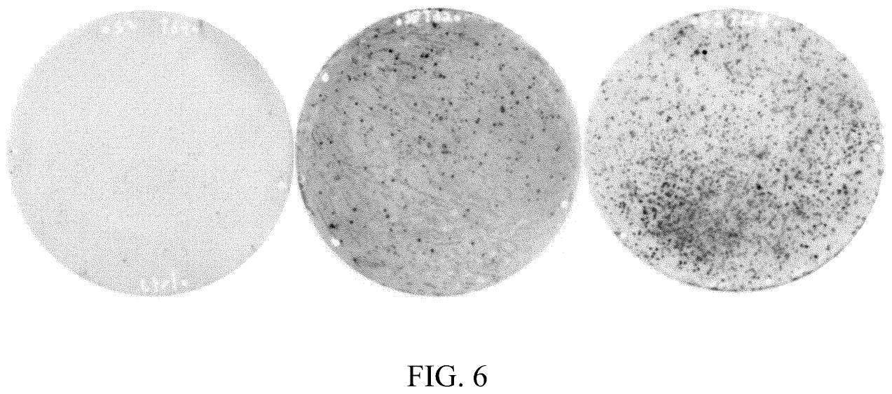

The round 2 image in FIG. 7 that the alpha fetoprotein signal from the 8 picked hits is greater than the alpha fetoprotein signal obtained from either the naïve library, the no chaperone control strain, or a positive control strain.

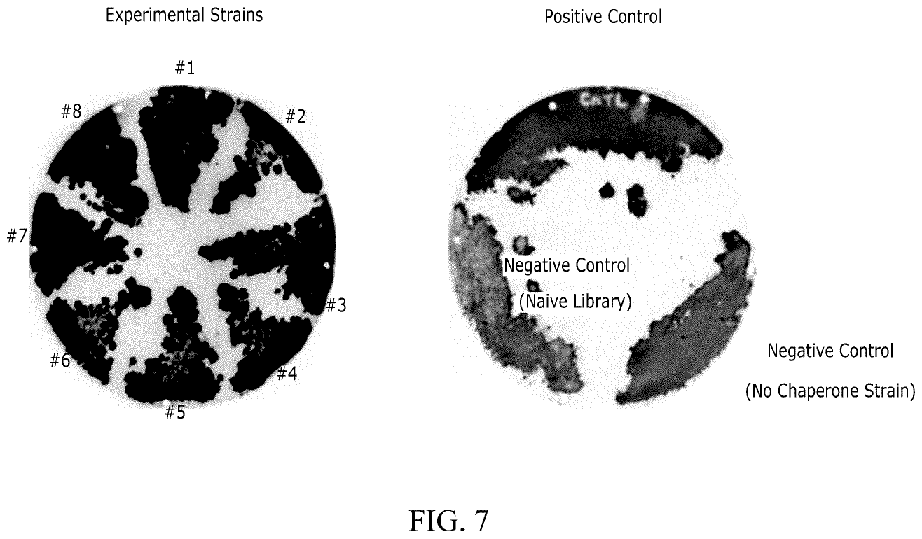

To ensure strain purity and a stable genotype, singles were picked from each dark sector from the round 2 image and passaged on LB Kanamycin (50 μg/ml) agar plate by streaking out for single colonies. The aim is to identify stable strains that improve alpha fetoprotein expression without any selective pressure (i.e. the presence of trimethoprim/sulfamethoxazole in the medium). After overnight growth, a repeat streak on LB kanamycin (50 μg/ml) agar was performed on a fresh plate. Well isolated single colonies after the second round of streak-purification were patched on a fresh, sectored induction plate, with each sector occupied by a single colony. The plate was incubated for 2 days and processed by solid phase as described above. The image of the processed membrane shown in FIG. 8 that 22 of the sectors are still positive for alpha fetoprotein expression, with the signal from many sectors exceeding the positive control strain.

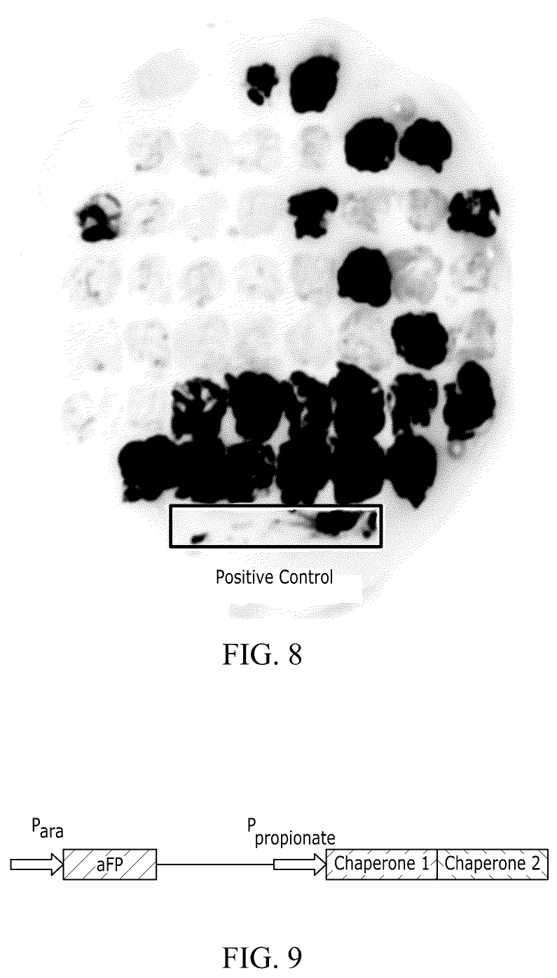

The plasmids from the 22 positive strains were recovered and analyzed. Twelve unique plasmids were identified. The folA gene was excised from these twelve plasmids and re-circularized to create new expression plasmids lacking folA but still harboring the original chaperones. (FIG. 9)

The new plasmids were transformed back into fresh host E. coli strain and newly created strains were streaked on fresh, sectored induction plates and incubated at 30° C. for 24 hours for analysis of alpha fetoprotein expression by solid phase assay.

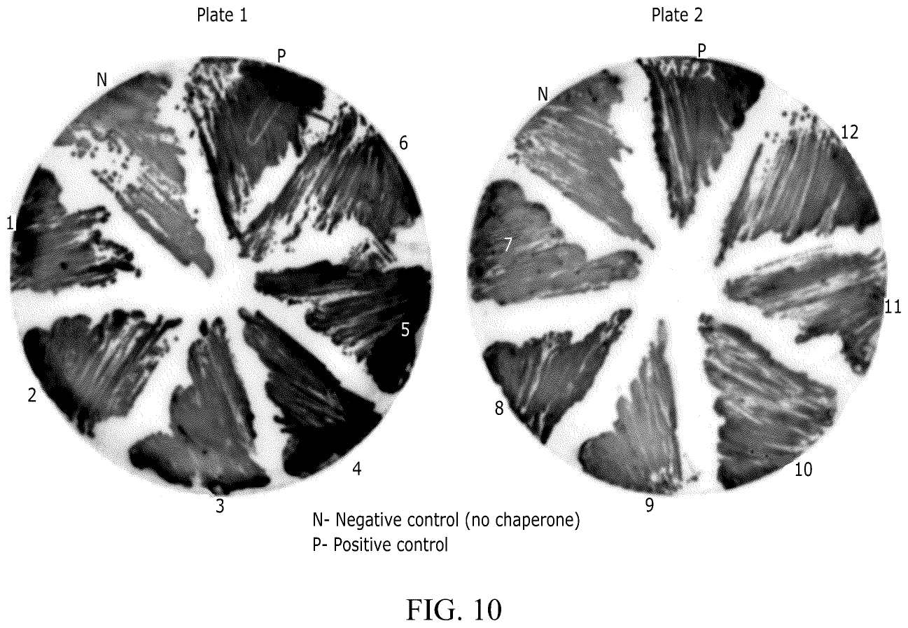

The solid phase assay showed that two (#4 & #5) of the newly created strains clearly expressed more alpha fetoprotein than the negative control (N, no chaperone) on agar plates. (FIG. 10) To determine whether these strains are outperformers in liquid culture, the same 12 strains were cultured in liquid induction medium containing 50 μg/ml kanamycin, 125 μM arabinose, 1 mM propionate. After 24 hrs of growth at 30° C., the cultures were harvested by pelleting the cells, and the soluble, monomeric alpha fetoprotein was quantitated by automated capillary Western analysis. The relative abundance of alpha fetoprotein was normalized to that present in the negative control strain (no chaperone).

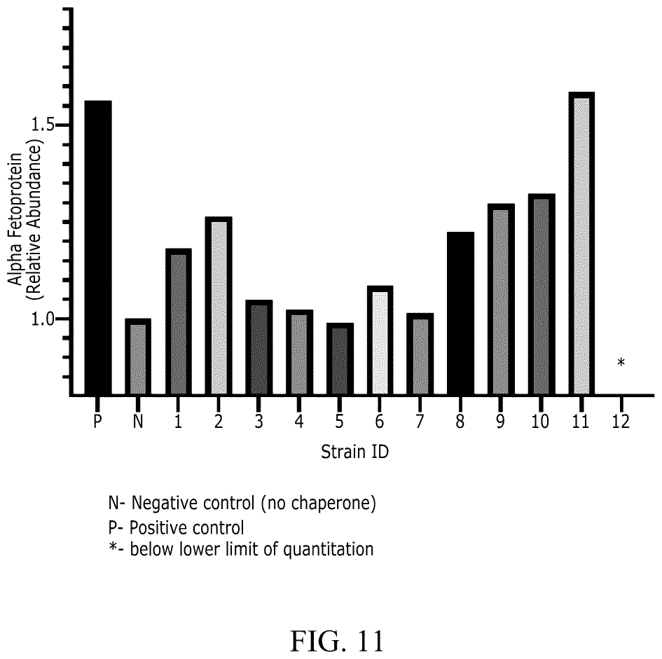

In liquid culture, six strains (#1,2,8,9,11) clearly expressed more alpha fetoprotein than the negative control strain (no chaperone). The outperformers in liquid culture are not the same as the two strains (#5 & 6) identified on agar plates. This finding is not unusual. (FIG. 11)

These data showed that the folA fusion assay, particularly when coupled in tandem with the solid phase assay, identifies higher performing strains in a pooled library.

The various embodiments described above can be combined to provide further embodiments. All U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified if necessary to employ concepts of the various patents, applications, and publications to provide yet further embodiments.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

What is claimed:

1. A method of identifying a host cell capable of producing a soluble protein of interest, said method comprising the steps of:
(a) preparing a population of host cells, wherein each host cell of the population comprises an expression construct capable of expressing a fusion protein comprising (i) a protein of interest and (ii) a selection protein;
(b) incubating the host cells of (a) under conditions that allow expression of the fusion protein, wherein said conditions comprise a growth substance comprising at least 2 synergistic selection agents; and
(c) visualizing host cells that are capable of growth;
thereby identifying capable of producing a soluble protein of interest.

2. A method of identifying host cells that produce the highest amounts of a soluble protein of interest among a population of host cells, said method comprising the steps of:
(a) preparing a population of host cells, wherein each host cell of the population comprises an expression construct capable of expressing a fusion protein comprising (i) a protein of interest and (ii) a selection protein;
(b) incubating the host cells of (a) under conditions that allow expression of the fusion protein, wherein said conditions comprise a growth substance comprising at least 2 synergistic selection agents; and
(c) visualizing host cells that are capable of growth;
thereby identifying host cells that produce the highest amounts of a soluble protein of interest among a population of host cells.

3. The method of any one of claims 1-2, further comprising the step of binning the host cells based on the amount of soluble protein produced.

4. The method of any one of claims 1-3, wherein the selection protein is a target of an antibiotic or an antibiotic resistance protein.

5. The method of claim 4, wherein the selection protein is FolA.

6. The method of claim 5, wherein the FolA is from E. coli.

7. The method of claim 6, wherein the FolA is set out in SEQ ID NO: 1.

8. The method of any one of claims 1-7 comprising 2 synergistic selection agents are used, wherein the synergistic selection agents are selected from the group consisting of antibiotics, sugars, chemical agents, enzyme substrate analogs, enzyme inhibitors, agents that sequester biomolecules, chelating agents, and agents that compromise the cell wall or cell membrane.

9. The method of claim 8 wherein the 2 synergistic selection agents are trimethoprim and sulfamethoxazole.

10. The method of any one of claims 1-9, wherein the protein of interest is a heterologous protein.

11. The method of claim 10, wherein the heterologous protein is selected from the group consisting of an antibody, a Fab, a scFv, a nanobody, a T cell receptor, and a chimeric antigen receptor, a growth factor, a cytokine, a hormone, an enzyme, or a functional fragment thereof.

12. The method of any one of claims 1-11, wherein the population of host cells comprises a library of host cells, wherein said library is comprised of cells with unique genotypes and/or that are uniquely genetically engineered.

13. The method of claim 10, wherein the library comprises approximately one thousand to approximately one billion host cells.

14. The method of any one of claims 1-13, wherein the host cells are selected from the group consisting of eukaryotic cells, prokaryotic cells, bacterial cells, mammalian cells and insect cells.

15. The method of claim 14, wherein the bacteria cells are E. coli cells.

16. The method of claim 15, wherein the E. coli cells comprise: (a) an alteration of gene function of at least one gene encoding a transporter protein for an inducer of at least one inducible promoter; (b) a reduced level of gene function of at least one gene encoding a protein that metabolizes an inducer of at least one inducible promoter; (c) a reduced level of gene function of at least one gene encoding a protein involved in biosynthesis of an inducer of at least one inducible promoter; (d) an altered gene function of a gene that affects the reduction/oxidation environment of the host cell cytoplasm; (e) a reduced level of gene function of a gene that encodes a reductase; (f) at least one expression construct encoding at least one disulfide bond isomerase protein; (g) at least one polynucleotide encoding a form of DsbC lacking a signal peptide; and/or (h) at least one polynucleotide encoding Ervlp.

17. The method of any one of claims 1-16, wherein the expression construct is an extrachromosomal construct selected from the group consisting of a polynucleotide, a plasmid, and an artificial chromosome.

18. The method of claim 17, wherein the expression construct comprises an inducible promoter.

19. The method of claim 17, wherein the expression construct comprises two or more inducible promoters.

20. The method of claim 19, wherein at least one inducible promoter is a propionate-inducible promoter and at least one other inducible promoter is an L-arabinose-inducible promoter.

21. The method of any one of claims 1-20, wherein the growth substrate is selected from the group consisting of a selective media.

22. The method of any one of claims 1-21, wherein the growth substrate comprises a matrix of 2 or more synergistic selection agents.

23. The method of claim 22, wherein the 2 synergistic selection agents are trimethoprim and sulfamethoxazole, and the agents are present in the growth media at a concentration range of 1 ug/ml to 1000 ug/ml and 1 ug/ml to 1000 ug/ml, respectively.

24. The method of any one of claims 1-23, wherein the conditions that allow expression of the fusion protein include the presence of one or more inducers of expression of the fusion protein.

25. The method of any one of claims 1-24, wherein the host cells are identified using a technique selected from the group consisting of visual inspection, chemiluminescence, radiography, fluorescence and colorimetric analyses.

26. The method of any one of claims 1-25, wherein the visualizing host cells in step (c) comprises detecting growth on agar plates.

27. The method of any one of claims 1-26 further comprising the steps of:
(a) plating the host cells on a growth substrate and incubating the host cells under conditions that allow host cell growth on the growth substrate;
(b) optionally preparing at least one replica plate and incubating said replica plate under conditions that allow host cell growth and production of the protein of interest;
(c) transferring host cells from the growth substrate or the at the least one replica plate of (b) to a membrane;
(d) preparing the host cells that have been transferred to the membrane in (c) for probing, comprising (i) optionally fixing the host cells under conditions that allow immobilization of cellular components; (ii) blocking the host cells; and (iii) optionally lysing the host cells under conditions that allow permeabilization of the host cells;
(e) contacting the permeabilized host cells with a probe solution comprising at least one probe under conditions that allow binding of the at least one probe the protein of interest, and thereby forming a probe-protein of interest complex; and
(f) imaging the host cells under conditions that allow identifying the host cell that produces the protein of interest.


SEQUENCE LISTING (1 sequence)

SEQ ID NO: 1 - protein, 159 aa, Escherichia coli [sequence omitted]